(19) United States
(12) Patent Application Publication
(10) Pub. No.: US 2022/0280507 A1
(43) Pub. Date: Sep. 8, 2022

(54) USES OF 5-METHYLTETRAHYDROFOLATE AND ITS COMPOSITION

(71) Applicant: Lianyungang Jinkang Hexin Pharmaceutical Co., Ltd., Jiangsu Province (CN)

(72) Inventors: Yong Zhi Cheng, Lianyungang (CN); Zeng Lin Lian, Beijing (CN)

(21) Appl. No.: 17/631,919

(22) PCT Filed: Jul. 30, 2020
(86) PCT No.: PCT/CN2020/106317

§ 371 Date: Feb. 1, 2022

(30) Foreign Application Priority Data
Aug. 2, 2019 (CN) 201910713858.5

(51) Int. Cl. A61K 31/4985; A61K 31/12; A61K 31/047; A61P 25/32; A61P 3/06; A61P 25/06; A23L 33/10

(52) U.S. Cl. CPC A61K 31/4985; A61K 31/12; A61K 31/047; A61P 25/32; A61P 3/06; A61P 25/06; A23L 33/10

(57) ABSTRACT

The present invention provides the uses of 5-methyltetrahydrofolate and its composition. The 5-methyltetrahydrofolate or its composition is used to treat, relieve or prevent diseases or symptoms caused by acute alcohol intoxication and chronic alcohol intoxication. The injuries or diseases caused by the acute alcohol intoxication include: headaches caused by drinking, negative emotions or depression caused by drinking, and hangover symptoms after drinking. The injuries or diseases caused by the chronic alcohol intoxication include: alcoholic fatty liver, central nervous system (CNS) inflammation, etc.

### FIELD OF THE INVENTION

The present invention belongs to the field of medicine, and relates to a pharmaceutical composition or health-care food composition containing 5-methyltetrahydrofolate, and its uses thereof.

### BACKGROUND OF THE INVENTION

Folic acid is vitamin B9, also known as pteroylglutamic acid (PGA). It has a variety of biochemical functions, such as promoting the maturation of juvenile cells in bone marrow. The lack of folic acid in humans can cause macrocytic anemia and leukopenia, so folic acid is important especially for pregnant women. As a nutritional supplement, 400 μg folic acid per day is recommended for adults, while consumption of folic acid exceeding the recommended dosage may cause side effects to the human body, since synthetic folic acid needs to be transformed into 5-methyltetrahydrofolate through complex human body before it enters blood circulation and participates in various physiological activities of the human body.

Folic acid, mainly present in blood and tissues of the human body in the form of 5-methyltetrahydrofolate (5-MTHF), participates in many biochemical reactions in the human body. Therefore, 5-MTHF is the main form of folic acid that works in the body, and can be directly absorbed and used by the body without complicated enzymatic reactions. Folic acid is absorbed in small intestine, and then reduced to dihydrofolic acid under the combined action of coenzyme reduced nicotinamide adenine dinucleotide phosphate (NADPH) and dihydrofolate reductase. Dihydrofolic acid is then reduced by reduced coenzyme II and ascorbic acid and metabolized to biologically active tetrahydrofolate (THF) which finally is reduced and methylated to 5-MTHF in the liver.

5-methyltetrahydrofolate has a chemical name of N-[4-[[(2-amino-1,4,5,6,7,8-hexahydro-4-oxo-5-methyl-(6S), -(6R), and (6R,S)-pteridyl)methyl)amino)toluoyl-glutamic acid, and can form salts in various forms.

5-methyltetrahydrofolate has become a new vitamin health product on the international market, which can serve as the main component of food additives and nutritional health products without any side effects and function well. Therefore, scientists have high enthusiasm and expectations for developing new uses of 5-methyltetrahydrofolate.

Drinking alcohol is an important behavior in human cultural and spiritual life, but long-term or one-time heavy drinking may cause great harm to the human body. Alcohol is mainly metabolized and detoxified by the liver. When it enters the human body, it will increase the burden on the liver, seriously affect normal liver function, and cause liver damage. In addition, alcohol has a strong stimulating effect on the heart, brain, and blood vessels and a negative impact on the nervous system of humans. Therefore, long-term or excessive drinking greatly increases the risk of cardiovascular and cerebrovascular diseases, and causes dizziness, headaches, migraines, nausea, drowsiness, anorexia, fuzzy cognition and other uncomfortable symptoms. The emotional state of people after drinking may also change, including many negative emotions, such as depression, and the mechanism for their occurrence is not clear.

There has been no clear definition of hangover for a long time. In 2016, at the 8th Alcohol Hangover Research Conference in New Orleans, USA, the expert group defined an alcohol hangover as “the combination of mental and physical symptoms, experienced the day after a single episode of heavy drinking, starting when blood alcohol concentration (BAC) approaches zero.” There are many theoretical explanations for alcohol hangovers, but these mechanisms cannot fully explain hangovers and have many limitations. Hangover is mainly explained from the following aspects: acute alcohol withdrawal, adverse reactions caused by acetaldehyde, electrolyte imbalance caused by alcohol, neuroinflammation, methanol in alcohol, and psychological factors.

The explanation that acute alcohol withdrawal causes hangovers is based on the existence of several common symptoms between them, such as nausea, vomiting, sweating, and anxiety [Swift R, Davidson D. Alcohol hangover: mechanisms and mediators. Alcohol Health Res World. 1998; 22(1): 54-60.]. However, hangovers may occur within a few hours after drinking and last for up to 24 h. Acute alcohol withdrawal symptoms occur within 1 to 5 days after stopping alcohol. In addition, acute alcohol withdrawal has symptoms that are not common with hangovers, such as hallucination and epilepsy. Therefore, it can be determined that hangover phenomena are different from withdrawal phenomena, and changes in hormones and hemodynamics during alcohol withdrawal are different from those in hangovers observed [Wiese J G, Shlipak M G, Browner W S. The alcohol hangover. Ann Intern Med. 2000; 132(11): 897-902.]. What's more, alcoholic withdrawal syndromes are produced by continuous heavy drinking for a long time, while people after a single episode of drinking or non-habitually drinking all have hangovers. Brain activity decreases during hangovers, while central nervous system over-excitement is observed during alcohol withdrawal [Fadda F, Rossetti Z L. Chronic ethanol consumption: from neuroadaptation to neurodegeneration. Prog Neurobiol. 1998; 56(4): 385-431.]. Therefore, although researchers have observed that alcoholics are prone to hangovers, they can determine that hangovers are different from alcohol withdrawal reactions.

The explanation with acetaldehyde also has a major drawback. Ethanol is metabolized into acetaldehyde under the action of alcohol dehydrogenase. Many researchers speculate that this alcohol metabolite causes hangovers. Aldehyde dehydrogenase (ALDH) rapidly metabolizes acetaldehyde into acetate. Although acetaldehyde is highly toxic, ALDH reacts very quickly. 36% of East Asians suffer from ALDH gene mutation leading to insufficient metabolism, so many Chinese may have facial flush and headaches due to drinking. This has led many people to believe that acetaldehyde causes hangovers. However, there are four points to be noted. 1. Even in the blood of patients with alcohol intoxication, the detected acetaldehyde content is very low. It is hard to detect acetaldehyde in the blood system of low-to-medium-dose drinking or acetaldehyde accumulation in the organism. 2. Few Europeans and Americans have ALDH gene mutations, but there are still many hangover cases. In China, people who drink without facial flush may also have hangovers. 3. Hangovers often occur on the next day after drinking and blood alcohol concentration has approached zero, but facial flush and headaches caused by acetaldehyde often occur during drinking. 4. Acetaldehyde is difficult to pass through blood-brain barrier, and few alcohol dehydrogenases present in the brain, resulting in much lower content of acetaldehyde in brain than that in blood, so it is difficult to reliably detect the production of acetaldehyde in brain. Therefore, hangovers are less closely related to acetaldehyde.

Some scholars believe that acetate is the main factor causing hangovers, largely because of an significant increase of acetate content in blood 6 hours after drinking, but acetate is difficult to pass through blood-brain barrier. In addition, acetate is produced in the anabolism of acetylcholine and some other metabolisms in the brain. There is no significant correlation between the incidence of hangovers and alcohol intake. Low to medium dose alcohol intake can still cause hangovers. Therefore, the theory is still flawed, or acetate may only be an influencing factor rather than the main factor causing hangovers.

There is a complex relationship between ethanol exposure and immune response. Ethanol can increase the expression of cytokines in the hippocampus and cortex. Inflammatory response may cause various symptoms, which may be the main factor causing hangovers. The levels of IL-12 and IFN-γ in human venous blood are significantly increased under the hangover condition. However, studies have shown [Marshall S A, McClain J A, Kelso M L, Hopkins D M, Pauly J R, Nixon K. Microglial activation is not equivalent to neuroinflammation in alcohol-induced neurodegeneration: The importance of microglia phenotype. Neurobiol Dis. 2013; 54: 239-251.] ethanol does not classically activate microglia, which does not meet the classical definition of inflammation. Alcohol-induced microglia activation is the result of alcohol-induced cell death. In particular, there has been no report on the presence of fully activated microglia in the brain of patients with alcohol intoxication. If microglia can only be partially activated, they may be beneficial for endogenous repair after alcohol-induced neurodegeneration.

Hangover symptoms may be caused by several homologues in alcoholic beverages, such as amine, amide, acetone, polyphenol, methanol and other substances. Methanol is found in high concentrations in red wine, and it is produced through the demethylation of pectin during fermentation. Methanol is considered as the main factor causing hangovers. National standards stipulate that the content of methanol in liquor made from grains shall not exceed 0.6 g/L, and that in liquor produced from other materials shall not exceed 2.0 g/L. (Methanol indexes are converted based on 100% alcohol content). It is well known that fruit wine made from fruits increases the frequency and intensity of hangovers compared with liquor made from grains. Studies have also shown that [Young-Sup, Woo, Su-Jung, el. Concentration changes of methanol in blood samples during an experimentally induced alcohol hangover state [J]. Addiction Biology, 2005.] changes in methanol concentration have a good correlation with changes in hangover subjective scale score. It should be noted that methanol itself is not toxic to human cells, but its oxidation products (formaldehyde and formate) are acute toxins, which are believed to cause damage to nerves. Ethanol can competitively inhibit methanol metabolism, and when the ethanol content in blood approaches zero, more methanol is metabolized and oxidized to formaldehyde or formate. The above explanation fits well with the time course of hangover symptoms, that is, hangover refers to physical and mental discomforts, generally starting on the next day when blood alcohol concentration approaches zero. Nevertheless, some data have been proposed against the explanation with methanol, such as [Mackus M, Van de Loo A J, Korte-Bouws G A, et al. Urine methanol concentration and alcohol hangover severity. Alcohol. 2017; 59:37-41.], showing no correlation between concentration of methanol in urine and severity of hangovers. Obviously, it is necessary to further study the correlation between methanol and hangovers.

People believed for a long time that methanol is an exogenous compound mainly derived from alcohol and some fruits. Researchers did not pay attention to the effects and influences of endogenous methanol and formaldehyde on the human body. With the improvement and advancement of testing methods, it was discovered recently that methanol and formaldehyde are actually natural compounds in normal healthy humans People have long observed that methanol content in blood increases after drinking, and alcohol manufacturers are trying to reduce the concentration of methanol in their products. However, recent studies have found that methanol content in blood increases rapidly even when people drink methanol-free alcoholic beverages. Researchers have observed that when people drink 50-90 ml of 40% ethanol (without methanol), the content of methanol in their blood still increases [Shindyapina A V, Pettrunia I V, Komarova T V, et al. Dietary methanol regulates human gene activity. PLoS One. 2014; 9(7): e102837.], and they believe that the metabolism of endogenous methanol is inhibited by ethanol, which leads to an increase in methanol content in blood.

Alcoholic liver disease is a type of liver disease caused by long-term or excessive alcohol consumption. In histopathology, it is divided into alcoholic fatty liver, hepatitis, liver fibrosis, and liver cirrhosis according to the process of liver cell fatty degeneration. The four diseases feature gradual processes, that is, different types of diseases can exist simultaneously. Research shows that alcoholic liver disease is a frequently-occurring and common disease among the population, and also the most serious complication of alcoholism.

Researchers have established the mechanism of correlation between alcohol intake and steatohepatitis, involving liver tissue damage caused by acetaldehyde metabolism, etc. However, it should be noted that only 30% of alcoholics may develop alcoholic hepatitis or other liver diseases [Grant B F, Dufour M C, Harford T C. Epidemiology of alcoholic liver disease. Semin Liver Dis. 1988; 8(1): 12-25.]. Therefore, another primary mechanism should be present to promote the progression of liver pathology in addition to the factor that large intake of alcohol can cause steatohepatitis. Intestinal endotoxin is the most likely influencing factor besides alcohol. Early studies have proved that the supplementation of oats can prevent gut leakiness and ameliorate alcohol-induced liver damage [Keshavarzian A, Choudhary S, Holmes E W, et al. Preventing gut leakiness by oats supplementation ameliorates alcohol-induced liver damage in rats. J Pharmacol Exp Ther. 2001; 299(2):442-448.], which has also been proved by much evidence, including use of antibiotics in animal models to prevent liver damage, protection of prebiotics on liver, etc. These studies have proved that the physiological function of intestine plays an important role in the pathogenic mechanism of liver injury.

Non-alcoholic fatty liver disease (NAFLD) is a clinicopathological syndrome caused by definite liver damage factors except alcohol, characterized by excessive fat accumulation in the hepatocytes. With the improvement of people's living standards, the incidence of NAFLD in China continues to increase. Clinically, the most common cause for the “rich man's disease” is the accumulation of fat in the liver caused by obesity and overnutrition. It is also common for people who regularly take folic acid to suffer from NAFLD.

According to the symptoms, NAFLD can be divided into non-alcoholic fatty liver (NAFL) in benign phenotypic state and non-alcoholic steatohepatitis (NASH). The proportion of NASH patients in overall NAFLD patients is about 44%, exhibiting pathological conditions characterized by lipid accumulation, inflammation, hepatocyte injury, and liver fibrosis. In severe cases, it can develop into liver cancer. The pathogenesis is complicated, and people still cannot fully understand the molecular pathogenesis of NASH. The pathogenesis may involve lipid metabolism disorders, insulin resistance, immune response, inflammation, oxidative stress, apoptosis, activation of hepatic stellate cells, and so on.

About 86.8% of health-care foods with auxiliary protection function for chemical liver injury published on the website of China Food and Drug Administration are products containing traditional Chinese medicines. As the components of traditional Chinese medicines are complex, it is difficult to distinguish whether any of them are hepatotoxic. Long-term administration may cause drug-induced liver injury.

### SUMMARY OF THE INVENTION

Through intensive research, the present invention has found that 5-methyltetrahydrofolate has new medicinal or auxiliary medicinal activities, and can relieve or treat various diseases caused by long-term or excessive drinking. Therefore, the present invention provides a pharmaceutical composition or health-care food composition containing 5-methyltetrahydrofolate, and new uses thereof. The technical scheme of the present invention is as follows:

A pharmaceutical composition or health-care food composition comprising an effective amount of 5-methyltetrahydrofolate, and the composition is used to treat or relieve diseases or symptoms caused by drinking or alcohol.

The composition according to the present invention, wherein it is used to treat, relieve or prevent injuries or diseases caused by acute alcohol intoxication.

The composition according to the present invention, wherein the injuries or diseases caused by acute alcohol intoxication include: headache caused by drinking, negative emotions or depression caused by drinking, and hangover symptoms after drinking.

The composition according to the present invention, wherein the hangover symptoms after drinking include symptoms such as headache, dizziness, fatigue, nausea, stomach upset, drowsiness, sweating, extreme thirst, and fuzzy cognition.

The composition according to the present invention, wherein it is used to treat, relieve or prevent injuries or diseases caused by chronic alcohol intoxication.

The composition according to the present invention, wherein the injuries or diseases caused by the chronic alcohol intoxication include: alcoholic fatty liver, central nervous system (CNS) inflammation, etc.

The composition according to the present invention, wherein the central nervous system inflammation includes migraines and headaches caused by it.

The composition according to the present invention, wherein it is used to treat or relieve non-alcoholic fatty liver disease. The non-alcoholic fatty liver disease includes non-alcoholic fatty liver (NAFL) and non-alcoholic steatosis hepatitis (NASH).

The composition according to the present invention, wherein it is used to shorten sobering up time.

The composition according to the present invention, wherein it is used to reduce risk of cardiovascular and cerebrovascular diseases caused by drinking.

The composition according to the present invention, wherein it also comprises pharmaceutically acceptable excipients or adjuvants.

In addition, the composition according to the present invention may also comprise other active compounds that act alone or synergistically.

The composition according to the present invention, wherein it comprises effective amounts of 5-methyltetrahydrofolate and curcumin

The above composition according to the present invention, wherein the mass ratio of 5-methyltetrahydrofolate to curcumin is 3˜1:1˜100. Exemplarily, the mass ratio is 3˜1:1˜3, such as 1:1.

The composition according to the present invention, wherein it can be made into various dosage forms known in the art. For example, enteral dosage forms, such as oral, sublingual or rectal administration. Exemplarily, oral dosage forms may be tablets, capsules, oral liquids, drops, pills, powders, and granules.

The composition according to the present invention, wherein the dosage of the 5-methyltetrahydrofolate in human is 5-50 mg/day and preferably 10-50 mg/day.

In a second aspect, the present invention provides a pharmaceutical composition or health-care food composition comprising an effective amount of 5-methyltetrahydrofolate, and the composition can significantly reduce the levels of total cholesterol (TC), triglycerides (TG), and malondialdehyde (MDA) in serum and increase superoxide dismutase (SOD) level.

The composition according to the present invention, wherein it can be used to treat or prevent hyperlipidemia and diseases caused by hyperlipidemia.

According to the present invention, the diseases caused by hyperlipidemia include: fatty liver, atherosclerosis, coronary heart disease, cerebral infarction, diabetes, vascular thrombosis, pancreatitis, etc.

In a third aspect, the present invention provides the uses of 5-methyltetrahydrofolate in the preparation of medicines or health-care food for treating, preventing or relieving the diseases described above.

### DETAILED DESCRIPTION OF THE INVENTION

In the present invention, the term “5-methyltetrahydrofolate” comprises 5-methyl-(6S)-tetrahydrofolate, 5-methyl-(6R)-tetrahydrofolate, 5-methyl-(6R, S)-tetrahydrofolate, that is, optical isomers of 5-methyltetrahydrofolate, especially pure optical isomers, mixtures of optical isomers, such as racemic mixtures, and physiologically acceptable salts thereof. Among them, 5-methyl-(6S)-tetrahydrofolate (also referred to as L-5-methyltetrahydrofolate) is particularly preferred.

The physiologically acceptable salts refer to acid addition salts converted from basic groups in 5-methyltetrahydrofolate. The acids can be inorganic acids, such as hydrochloric acid, sulfuric acid, nitric acid, and phosphoric acid; organic acids, such as formic acid, acetic acid, propionic acid, diethylacetic acid, propanedioic acid, succinic acid, fumaric acid, maleic acid, lactic acid, tartaric acid, malic acid, citric acid, gluconic acid, ascorbic acid or nicotinic acid, etc.

The physiologically acceptable salts can also refer to base addition salts converted from acidic groups in 5-methyltetrahydrofolate, appropriately, such as sodium, potassium, magnesium, calcium and ammonium salts, substituted ammonium salt, and salts formed with arginine or lysine.

The term of “acute alcohol intoxication” in the present invention is a general term in the art, commonly known as drunkenness. It is a disease with many neuropsychiatric symptoms caused by a single episode of drinking excess alcohol or alcoholic beverages, often presented with abnormal behavior and consciousness. Patients with it generally present in three clinical stages. \textcircled{1} Stage I of excitation, exhibiting headaches, dizziness, euphoria, excitement, lerema, emotional instability, irritability, or aggressive behavior, and a few can be silent and withdrawn. \textcircled{2} Stage II of ataxia, exhibiting uncoordinated movements, incoherent speech, nystagmus, turbulence, blurred vision, diplopia, nausea, vomiting, and drowsiness. \textcircled{3} Stage III of lethargy (or coma), exhibiting lethargy, pale face, skin clamminess, slightly cyanotic lips, and deep coma in severe cases, manifested as moderately dilated pupils, increased heart rate, decreased blood pressure, urinary and fecal incontinence, possibly deaths due to respiratory and circulatory failure, or aspiration pneumonia or suffocation caused by vomiting after meals due to weakened pharyngeal reflex.

The term of “hangover” in the present invention refers to mental and physical discomforts remained, experienced the day after a single episode of heavy drinking, starting when blood alcohol concentration (BAC) approaches zero, such as headaches, dizziness, fatigue, nausea, stomach upset, drowsiness, sweating, extreme thirst, and fuzzy cognition. Currently, scientists believe that the neural mechanisms of hangover are related to neuroinflammatory factors, changes in neurotransmitters and receptors, mitochondrial dysfunction, and alcohol metabolites. Some scholars hold that inflammatory factors, changes in neurotransmitters and receptors, and mitochondrial dysfunction are the most likely contributing factors to the pathology of hangover [Palmer E, Tyacke R, Sastre M, Lingford-Hughes A, Nutt D, Ward R J. Alcohol Hangover: Underlying Biochemical, Inflammatory and Neurochemical Mechanisms. Alcohol Alcohol. 2019; 54(3): 196-203.]. However, none of the explanations above can explain the mental and physical discomforts during the time course (a few hours or the day after drinking) of a hangover after the human blood alcohol concentration drops to nearly zero.

After drinking, the elimination of methanol from the blood lags behind and that of ethanol lasts about 6 to 8 hours. Data shows that the blood methanol concentration continues to increase within a few hours after drinking. The main reason is that the methanol metabolism system beyond the brain is inhibited by ethanol, and endogenous methanol is produced in the human body, resulting in a certain methanol accumulation. Endogenous methanol is produced at least about 1.66 mg/kg/h, and 116 mg of methanol can be produced in the body of an adult within 1 hour, equivalent to 250-300 ml red wine intake. This shows that the endogenous production of methanol is not negligible.

In an embodiment of the present invention, the contents of methanol and formaldehyde increase sharply in the urine of rats after acute drinking. The concentration of methanol reaches a peak 8 hours after drinking, while the peaks of formaldehyde and formate fall behind that of methanol. It should be noted that the concentration of formate is tens or hundreds of times higher than that of formaldehyde and methanol. As the ability of rats to metabolize methanol far exceeds that of humans, formic acid is more commonly accumulated in humans and monkeys. The accumulation of endogenous methanol, formaldehyde, and formic acid should be more serious in humans after drinking alcohol. The inventors review the existing and fragmentary information on hangovers, showing that there are many explanatory hypotheses about hangovers. Such hypotheses were proposed based on the potential direct impact of alcohol drinking or the impact of alcohol withdrawal, and almost all the explanations are flawed. As described in the background of the invention, people tend to focus on alcohol metabolites rather than the influence of alcohol on the metabolism of endogenous methanol and formaldehyde. The experimental results of the present invention show that formaldehyde and formate accumulate in the brain after drinking. It is well known that formaldehyde and formate are toxic to nerves. Formaldehyde plays a certain role in the pathological development of brain dysfunction. Methanol, formaldehyde and formate metabolized therefrom are probably the most important factors for hangovers, which is supported by the data of the present invention.

In another embodiment of the present invention, the observation on the contents of formaldehyde, formate and some neurotransmitters (5-hydroxytryptamine, dopamine) in brain of rats 24 hours after drinking alcohol surprisingly shows an accumulation of formaldehyde and formate in brain: the concentrations of formaldehyde and formate in urine of rats have basically dropped to the baseline level in about 24 hours, while the contents of formaldehyde and formic acid in brain tissue are still high. Methanol and ethanol can easily pass through blood-brain barrier and act on the brain, while formaldehyde and acetaldehyde are difficult to pass through blood-brain barrier. Moreover, ADH1 is not active in the brain, so the production of endogenous formaldehyde decreases in the brain. Formaldehyde produced from methanol by oxidation in brain is rapidly detoxified by enzymes such as FDH and ALDH2. Under normal circumstances, the above two strategies can protect central neurons from the harm of formaldehyde. However, the above two strategies are challenged during acute drinking, especially a decrease in the content of SOD and GSH in brain tissue after drinking increases the possibility of oxidation of methanol to formaldehyde in the brain. In addition, ethanol as a dehydrating agent can also cause the increase of formaldehyde in brain tissue. The present invention has found that 5-methyltetrahydrofolate can promote the metabolism of formaldehyde and formate in brain tissue, and reduce the concentrations of formaldehyde and formate, thereby preventing and treating hangovers.

The negative emotions proposed in the present invention refer to low mood, sense of loss, loss of appetite, insomnia, susceptibility to fatigue, inattention, loss of interest in surrounding things, self-reinforcing feelings of guilt or disgust, etc. If such emotions persist and are accompanied by severe depressed mood, depression would be developed. Some drinkers experience the above negative emotions after drinking, or drinking aggravates the symptoms of patients originally with the above negative emotions or depression. In an embodiment of the present invention, the level of 5-hydroxytryptamine in the brain tissue of rats decreases 24 h after drinking, which may be the cause of depression after drinking, and 5-methyltetrahydrofolate can improve the secretion of 5-hydroxytryptamine.

The chronic alcoholic intoxication (alcohol dependence) proposed in the present invention, also a general technical term in the art, refers to severe central nervous system toxicity caused by chronic excessive drinking, manifested as a thirst for alcohol and a compulsive experience of frequent need to drink, showing withdrawal symptoms such as feeling unwell, restless, or limb tremor, nausea, vomiting, and sweating after stopping drinking, and quick disappearance of such symptoms when re-drinking. Due to chronic alcohol ingestion, most symptoms are combined with somatic damages apparently to the heart, liver, and nervous system, most commonly liver damage, peripheral neuropathy and epileptic seizures, and some form ethyl alcohol toxic dysphrenia and alcoholic-toxic encephalopathy.

The alcoholic fatty liver disease proposed in the present invention is hepatic lesions caused by chronic alcoholic intoxication, i.e. chronic alcohol ingestion. Fatty liver can be roughly divided into alcoholic fatty liver disease (AFLD) and non-alcoholic fatty liver disease (NAFLD). NAFLD is a disease characterized by inflammation and fibrosis showing fatty degeneration of liver parenchymal cells and fat accumulation without a history of excessive drinking. It is closely related to the occurrence of obesity and hyperlipidemia. NAFLD was defined as hepatic steatosis confirmed by imaging and liver histology, and excluding other causes of hepatic steatosis, such as heavy drinking, long-term use of lipogenic drugs, or single-gene genetic disorders. NAFLD is divided into NAFL and NASH according to liver histological changes (1) NAFL: More than 5% fatty change of liver cells, without ballooning change of hepatocytes. (2) NASH: More than 5% fatty change of liver cells, accompanied by inflammation and hepatocyte damage (such as ballooning change), with or without fibrosis. The stage of liver fibrosis (stage, S) is S3 (bridging fibrosis) and S4 (cirrhosis), defined as advanced liver fibrosis.

The susceptibility of alcoholic fatty liver to alcoholic steatohepatitis is highly variable. The reason why some people suffer from alcoholic steatohepatitis while other people still have benign fatty liver after chronic alcohol ingestion involves amount, time, age, gender, race, complications, nutritional status and environment of alcoholism. In addition, genetic and epigenetic factors can also lead to differences in susceptibility to alcoholic steatohepatitis. Recent evidence has shown the role of intestinal microbiota and its metabolites in the pathological development of alcoholic steatohepatitis [Meroni M, Longo M, Dongiovanni P. Alcohol or Gut Microbiota: Who Is the Guilty. Int J Mol Sci. 2019; 20 (18): 4568.]. Specifically, the increase in intestinal permeability due to alcohol abuse leads to an increase in the concentration of lipopolysaccharide (LPS) entering the portal blood stream. As a result, LPS binds to the Toll-like receptor 4 (TLR4) of liver tissue and activates activated B Cells and NF-κB to release pro-inflammatory cytokines, which contributes to conversion of benign fatty liver into steatohepatitis. It has been found that alcohol-fed mice without alcoholic hepatitis exhibit severe liver inflammation and necrosis after being transplanted with intestinal microbiota isolated from patients with alcoholic hepatitis [Llopis M, Cassard A M, Wrzosek L, et al. Intestinal microbiota contributes to individual susceptibility to alcoholic liver disease [J]. Gut, 2016, 65(5): 830-839.].

Several experimental models have shown that endotoxemia and alcoholic hepatitis can be prevented by antibiotics, but the long-term administration of antibiotics is controversial, including bacterial resistance caused by abuse of antibiotics, hepatotoxicity of drug itself, and destruction of intestinal microbial ecosystem. However, the above studies have also proved that intestinal microbes are closely related to the pathogenesis of alcoholic fatty liver and its progression to hepatitis. In an embodiment of the present invention, 5-methyltetrahydrofolate can significantly protect intestinal barrier function of and reduce serum endotoxin level. The result shows that 5-methyltetrahydrofolate can prevent the production of alcoholic “permeable intestine” thus to reduce the flow of endotoxins into enterohepatic circulation to further prevent and treat alcoholic steatohepatitis. The mechanism discovered may also be applicable to the prevention of non-alcoholic steatohepatitis with 5-methyltetrahydrofolate.

In addition, increasing evidence shows that chronic alcoholic intoxication affects glial cells, and is closely related to central nervous system (CNS) inflammatory response, with the main manifestation of neuronal damage caused by activated phagocytes (microglia) and other glial cells in the brain through paracrine pathways. In vitro studies have shown that chronic alcoholic intoxication can inhibit the expression of glial fibrillary acidic protein and S100 protein in brain astrocytes. Alcohol can induce apoptosis and necrosis of astrocytes by damaging the cell membrane and mitochondria of brain astrocytes.

The inventors have found that serum folic acid concentration drops sharply in a short time after a single episode of heavy drinking, even below quantitative lower limits, which is beyond that to be expected in the art. Although the liver itself stores folic acid, this portion of folic acid cannot immediately enter the bloodstream after drinking. Even if supplemented in advance or after drinking, folic acid needs to be converted into tetrahydrofolic acid in the liver by dihydrofolate reductase. However, the activity of this enzyme in human liver is low, and more than 0.2 mg of folic acid leads to saturation of liver metabolism [Bailey, Steven, W, el. The extremely slow and variable activity of dihydrofolate reductase in human liver and its implications for high folic acid intake. [J]. Proceedings of the National Academy of Sciences of the United States of America, 2009.]. Therefore, the supplementation of folic acid cannot solve the decrease of serum folic acid concentration, but the 5-methyltetrahydrofolate or its composition provided in the present invention can solve the acute decrease of serum folic acid concentration caused by a single episode of heavy drinking.

Based on the above findings, the present inventors have further confirmed through experiments that the administration of 5-methyltetrahydrofolate or its composition can prevent, improve, and treat various discomforts caused by acute alcohol intoxication in humans or animals The discomforts include hangovers, headaches, and negative mental states after drinking.

In addition, the daily dosage of folic acid as a supplement for pregnant women is 0.4 mg in China, and the U.S. Food and Drug Administration has mandated that 140 ug of folic acid must be added to 100 g of grain products since 1996. If one person consumes 500 g of grain products every day, he should supplement with folic acid at a dosage of 0.7 mg/day. However, the national campaign of folic acid supplement based on such dosage has not reduced the above diseases caused by acute or chronic alcohol intoxication. The incidence of fatty liver in the United States continues to increase year by year, and is currently more than 15%. And an document [Christensen K E, Mikael L G, Leung K Y, et al. High folic acid consumption leads to pseudo-MTHFR deficiency, altered lipid metabolism, and liver injury in mice. Am J Clin Nutr. 2015; 101(3): 646 -658.] shows that high folic acid intake can cause liver damage and promote liver cell apoptosis. Although 5-methyltetrahydrofolate is a metabolite of folic acid, the two are essentially different.

The inventors have found that a certain dose of 5-methyltetrahydrofolate or its composition can prevent, improve, and treat various diseases in humans or animals caused by chronic alcoholic intoxication. Manufacturers of existing anti-alcohol health-care products or medicines mainly focus on the metabolism of alcohol by liver rather than the metabolism of alcohol homologues (methanol, formaldehyde, formate) produced from drinking by brain tissue. The present invention can promote the metabolism and elimination of related substances in brain tissue.

In an embodiment of the present invention, 5-methyltetrahydrofolate can inhibit the expression of cerebral inflammatory factors (such as TNF-α, IL-1β) caused by chronic alcoholic intoxication, and has a certain dose-effect relationship. Therefore, 5-methyltetrahydrofolate can prevent headaches and migraines caused by chronic alcoholic intoxication.

In an embodiment of the present invention, 5-methyltetrahydrofolate exhibits its ability to improve the activity of alcoholic liver injury caused by long-term drinking (i.e. chronic alcoholic intoxication). It significantly reduces the levels of total cholesterol (TC), triglyceride (TG), malondialdehyde (MDA) in serum and increases the level of superoxide dismutase (SOD), and can restore liver to its normal form with good quality, index, and histopathological observation results. In addition, 5-methyltetrahydrofolate can maintain intestinal barrier function and reduce the flow of intestinal endotoxins into hepatoenteral circulation, indicating that 5-methyltetrahydrofolate can prevent alcoholic fatty liver from deteriorating into diseases such as hepatitis, especially it can prevent and treat alcoholic fatty liver, hepatitis, and liver fibrosis.

In an embodiment of the present invention, 5-methyltetrahydrofolate exhibits the activity of preventing or treating fatty liver caused by hyperlipidemia, thereby preventing or treating hyperlipidemia. It is well known to those skilled in the art that hyperlipidemia can cause a series of related diseases. Studies have shown that it is closely related to the onset of fatty liver, atherosclerosis, coronary heart disease, cerebral infarction, diabetes, vascular thrombosis, pancreatitis, etc.

In an embodiment of the present invention, the 5-methyltetrahydrofolate exhibits the activity of improving bad mental state, negative emotions, and even depression after drinking too much alcohol at one time (acute alcohol intoxication).

In an embodiment of the present invention, the 5-methyltetrahydrofolate exhibits an effect of inhibiting the release of inflammatory factors from neuroglia cells induced by lipopolysaccharide. More surprisingly, the combined use of 5-methyltetrahydrofolate and curcumin plays a synergistic role in inhibiting lipopolysaccharide-induced release of inflammatory factors from neuroglia cells, especially the inflammatory factor of TNF-α. When used in combination, the ratio of 5-methyltetrahydrofolate to curcumin can be 3˜1:1˜3, such as 1:1.

The present invention provides a medicine or health-care food, each dose of which contains 1-500 mg of 5-methyltetrahydrofolate. Preferably, a medicine or health-care food containing 0.1-100 mg active substance per dose is used in preventive medication, and that containing 5-200 mg of active substance per dose is used in therapeutic medication. The specific dosage depends on various factors including patient's age, weight, time and route of administration, condition, etc. Preferably, the optimal therapeutic dosage is 5-50 mg/day, such as 10-50 mg/day, and the preventive dosage is 1-10 mg/day.

In the present invention, the pharmaceutical composition or health-care food composition contains various carriers, excipients and/or adjuvants, such as water, oil, benzyl alcohol, polyethylene glycol, glycerol triacetate, gelatin, lecithin, cyclodextrin, lactobiose or starch and other carbohydrates, magnesium stearate, talc, silica gel or cellulose. The adjuvants are stabilizers, antioxidants, buffers, bacteriostats, etc. In an embodiment of the present invention, the excipient or carrier is microcrystalline cellulose, or a combination of microcrystalline cellulose and croscarmellose sodium, or superfine silica powder.

### BRIEF DESCRIPTION OF THE DRAWINGS

FIG. 1 is a diagram of liver tissue of rats in normal group in Experiment 4.

FIG. 2 is a diagram of liver tissue of rats in model control group in Experiment 4.

FIG. 3 is a diagram of liver tissue of rats in positive drug group in Experiment 4.

FIG. 4 is a diagram of liver tissue of rats in low dose group in Experiment 4.

FIG. 5 is a diagram of liver tissue of rats in medium dose group in Experiment 4.

FIG. 6 is a diagram of liver tissue of rats in high dose group in Experiment 4.

FIG. 7 is an ion chromatogram and an internal standard ion chromatogram of MR1 without administration in Experiment 6.

FIG. 8 is a broken line graph of urine formaldehyde average concentration and time in each group of rats after drinking in Experiment 10.

FIG. 9 is a broken line graph of urine formic acid average concentration and time in each group of rats after drinking in Experiment 10.

FIG. 10 is a diagram of oral dose fraction (small intestine permeability) of 5-hour urine lactulose in each group of alcohol-fed rats at week 8 in Experiment 12.

FIG. 11 is a diagram of oral dose fraction (permeability of whole intestine [small intestine+large intestine]) of 5-hour urine sucralose in each group of alcohol-fed rats at week 8 in Experiment 12.

FIG. 12 is a diagram of serum endotoxin levels in each group of alcohol-fed rats at week 8 in Experiment 12.

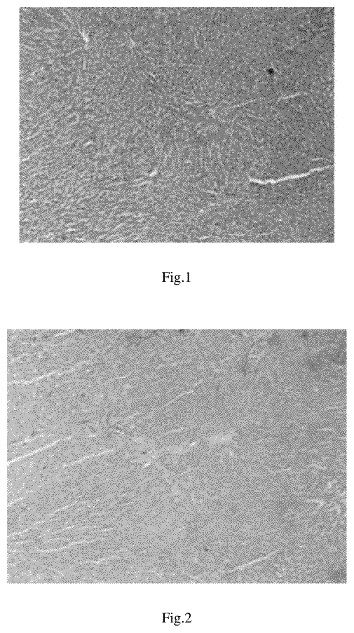

### DETAILED DESCRIPTION OF PREFERRED EMBODIMENTS

Special Notes:

The serum folic acid proposed in the present invention refers to 5-methyltetrahydrofolate in the serum.

Folic acid, unless otherwise specified, refers to synthetic folic acid.

### Embodiment 1

100 g of 5-methyltetrahydrofolate calcium salt was mixed with 700 g of microcrystalline cellulose. After dry granulation, 1000 capsules were filled to make capsule preparations containing 100 mg of 5-methyltetrahydrofolate calcium each.

### Embodiment 2

200 g of superfine silica powder was added to 100 g of 5-methyltetrahydrofolate calcium salt, mixed evenly and compressed by tablet machine to form antialcoholic lozenges.

### Embodiment 3

The weight of each bulk drug is as follows: 40 g of 5-methyltetrahydrofolate calcium, and 40 g of curcumin The bulk drugs were crushed and mixed with microcrystalline cellulose and croscarmellose sodium to make granules, then dried and filled to obtain capsules containing 20 mg of 5-methyltetrahydrofolate calcium and 20 mg of curcumin each.

The weight of each bulk drug is as follows: 40 g of 5-methyltetrahydrofolate calcium, 20 g of reduced glutathione particles, and 40 g of curcumin. The bulk drugs were crushed and mixed with microcrystalline cellulose and croscarmellose sodium to make granules, then dried and filled to obtain capsules containing 20 mg of 5-methyltetrahydrofolate calcium, 10 mg of reduced glutathione particles and 20 mg of curcumin each.

Experiment 1 Effect of 5-methyltetrahydrofolate on disappearance and recovery of righting reflex of alcohol drinking rats.

30 SPF-grade SD rats were chosen in half male and half female, and divided into 5 groups with 6 rats in each group, including normal group, model group, positive drug group (Weihe Liver Protectant), 5-methyltetrahydrofolate administration group (4 mg·Kg−1 medium dose group, 8 mg·Kg−1 high dose group, respectively). After fasting for 12 h, the administration group was given Weihe Liver Protectant at 50 mg·kg−1, 5-methyltetrahydrofolate (Jinkang Hexin product) at 4 mg·Kg−1, 5-methyltetrahydrofolate (Jinkang Hexin product) at 8 mg·Kg−1, respectively. After 30 min, the model group and the administration group were given Erguotou (batch number: 201603092, specification: 2L, place of origin: Beijing Shunxin Agriculture Co., Ltd.) at 9 ml·Kg−1, and the normal group was given the same amount of normal saline. The intoxication in rats depends on righting reflex disappearing, that is, cover the head of the rat with gauze, and gently place the rat back down in the animal cage. If the front paw of the rat turns back within 1 min, it shows that no righting reflex has occurred, otherwise righting reflex has occurred. The disappearance time and recovery time of the righting reflex in rats after drinking were recorded. The experimental results are shown in the table below.

TABLE 1
Effect of 5-methyltetrahydrofolate on the time of righting
reflex in rats (x̅ ± s)

[symbol]

Note:
n = 6, compared with the model group: *p < 0.05, **p < 0.01 [symbol]
indicates data missing or illegible when filed

The above results indicate that 5-methyltetrahydrofolate has a significant antagonistic effect on rats with acute alcohol intoxication. Where, the recovery time of righting reflex of alcohol drinking rats in the medium and high dose groups with much lower dosage than the positive drug group is better than that of the positive drug group, which shows that 5-methyltetrahydrofolate has the effect of shortening the sober-up time.

Experiment 2 Effect of 5-methyltetrahydrofolate on homocysteine (Hcy) in plasma of alcohol drinking rats.

30 SPF-grade SD rats were chosen in half male and half female, and divided into 5 groups with 6 rats in each group, including normal group, model group, positive drug group (Weihe Liver Protectant), 5-methyltetrahydrofolate administration groups (4 mg·Kg−1 medium dose group, 8 mg·Kg−1 high dose group, respectively). After fasting for 12 h, the administration groups were given Weihe Liver Protectant at 50 mg·kg−1, 5-methyltetrahydrofolate (Jinkang Hexin product) at 4 mg·Kg−1, 5-methyltetrahydrofolate (Jinkang Hexin product) at 8 mg·Kg−1, respectively. After 30 min, the model group and the administration groups were given Erguotou (batch number: 201603092, specification: 2L, place of origin: Beijing Shunxin Agriculture Co., Ltd.) at 20ml·Kg−1, and the normal group was given the same amount of normal saline. 6 h after drinking, blood was collected from the eye socket, treated with 0.5% EDTA-Na for anticoagulation (10 ml/L), immediately cooled in an ice bath, and centrifuged at 4° C. (3500r/min) for 15 min within 30 min. Plasma was collected and stored at −20° C. The plasma Hcy concentration was determined with high pressure liquid chromatography-fluorescence detection method. The experimental results are shown in the table below.

TABLE 2
Effect of 5-methyltetrahydrofolate on homocysteine in plasma
of alcohol [symbol] (x̅ ± s) rats.

| | | | Disappearance | Recovery |
|---|---|---|---|---|
| Group | Dosage | | time/min | time/min |
| Normal group | / | | / | / |
| Model group | / | | 17.4 ± 9.6 | 143.2 ± 13.5 |
| Positive drug | 50 | mg · Kg⁻¹ | 37.4 ± 8.2** | 108.5 ± 14.5* |
| group | | | | |
| 5-methyltetrahydrofolate | 4 | mg · Kg⁻¹ | 30.1 ± 14.4* | 78.5 ± 14.5* |
| (medium) | | | | |
| 5-methyltetrahydrofolate | 8 | mg · Kg⁻¹ | 42.3 ± 15.6** | 64.1 ± 14.5** |
| (high) | | | | |

| | | | Hcy |
|---|---|---|---|
| Group | Dosage | | (μmol/L) |
| Normal control | — | | 2.52 ± 0.89 |
| group | | | |
| Positive drug | 50 | mg · Kg⁻¹ | 5.53 ± 1.26 |
| group | | | |
| Model group | — | | 5.58 ± 2.23 |
| Medium dose | 4 | mg · Kg⁻¹ | 3.13 ± 1.26** |
| group | | | |
| High dose | 8 | mg · Kg⁻¹ | 2.32 ± 1.49** |
| group | | | |

Note:
n = 6, compared with the model group: *p < 0.05, **p < 0.01 [symbol]
indicates data missing or illegible when filed

The results show that acute heavy alcohol intake can cause an increase in homocysteine in rats, and 5-methyltetrahydrofolate can significantly reduce the level of Hcy in serum of rats, and has a dose-effect relationship. Hcy may not only cause damage to the nervous system, but also cause ischaemic heart disease and ischemic stroke. Although these indexes return to normal levels with alcohol metabolism, bad conditions still be produced. Therefore, 5-methyltetrahydrofolate can also reduce the risk of cardiovascular diseases caused by alcohol drinking.

Experiment 3 Protection of 5-Methyltetrahydrofolate Calcium on Liver Injury Induced by Carbon Tetrachloride

30 SPF-grade SD rats were chosen in half female and half male and randomly divided into vehicle control group (vegetable oil), model control group (1.5 ml·kg−1, 20% carbon tetrachloride vegetable oil solvent, intraperitoneal injection every 3 days), 5-methyltetrahydrofolate calcium low-dose group (1mg·Kg−1), 5-methyltetrahydrofolate calcium medium-dose group (2 mg·Kg−1), and 5-methyltetrahydrofolate calcium high-dose group (4 mg·Kg−1) according to their body weight. 20% carbon tetrachloride vegetable oil solvent was used as the inducer, and intraperitoneal injection was performed every 3 days for a total of 60 days. After successful modeling, the administration groups were given a dosage of 5-methyltetrahydrofolate calcium as prescribed above for gavage once a day, and the model group and the normal control group were gavaged once daily with purified water for 60 consecutive days. Blood was taken on the 61st day, and the levels of ALT and AST in serum were detected. The results are shown in the table below.

TABLE 3
Protection of 5-methyltetrahydrofolate calcium
on liver injury induced by carbon tetrachloride

| | Dosage | ALT | AST |
|---|---|---|---|
| Group | (mg · kg⁻¹ · d⁻¹) | (IU/L) | (IU/L) |
| Normal control | — | 26.34 ± 12.31 | 87.29 ± 35.25 |
| group | | | |
| Model group | — | 182.56 ± 67.37* | 382.32 ± 87.94* |
| Low dose | 1 | 54.64 ± 20.52^{#} | 185.73 ± 63.46^{#} |
| group | | | |
| Medium dose | 2 | 50.54 ± 20.12^{#} | 156.97 ± 50.35^{#} |
| group | | | |
| High dose | 4 | 40.84 ± 16.31^{#} | 125.87 ± 43.84^{#} |
| group | | | |

Note:
1. Number of animals: n = 6 in each group;
2. *Compared with the vehicle control group, each dose group, P < 0.05; ^{#}Compared with the model control group, in each dose group, P < 0.05.

Experiment 4 Prevention of a Certain Dose of 5-Methyltetrahydrofolate Calcium on Alcoholic Fatty Liver Damage

96 SPF-grade SD rats were chosen in half female and half male with an average weight of 176-220 g at the beginning of the experiment. According to their weight, they were randomly divided into vehicle control group (purified water, ig), model control group (liquor, 10 ml·Kg−1), positive control group (bicyclol, 50 mg·Kg−1, ig), folic acid calcium salt high-dose group (4 mg·Kg−1), folic acid calcium salt medium-dose group (2 mg·Kg−1), folic acid calcium salt low-dose group (1 mg·Kg−1), in half female and half male, and 16 rats in each group. Purified water was used as a negative control, and bicyclol (place of origin: Beijing Xiehe Pharmaceutical Factory, specification: 25 mg/tablet, batch number: H20040467) was used as a positive control drug. Route of administration: all were administered by oral gavage. Dosage of administration: 1 mg·Kg−1, 2 mg·Kg−1, and 4 mg·Kg−1. Frequency of administration: once a day for 60 consecutive days. Erguotou (batch number: 201603092, specification: 2L, place of origin: Beijing Shunxin Agriculture Co., Ltd.) model establishing method: gavage with diluted 56-degree Erguotou twice a day for 60 consecutive days.

Method of administration: administration started at prescribed doses on the day of modeling for 60 consecutive days and ended at the 60th day. At the same time, rats in each group were weighed every other week during administration period to monitor their body weight changes. The changes in the weight gain of rats during administration period are shown in Table 4. On the 61st day, rats were dissected and serum was collected for determination of triglyceride (TG), total cholesterol (TC), malondialdehyde (MDA), superoxide dismutase (SOD) and liver histopathologic examination. The results are shown in Table 5. The brain tissue of rats was taken out, and hippocampus tissue was separated on ice plate, prepared to homogenate with a mass fraction of 10% with purified water, then the homogenate was centrifuged to obtain supernatant. ELISA was used to detect the levels of inflammatory factors TNF-α and IL-1β in hippocampus tissue with continuous spectrum scanning microplate reader. The results are shown in Table 6.

TABLE 4
Changes in the weight gain of rats during the administration period⁽^̅{̅x̅}̅± s)

| | Dosage | Weight gain (g) | | | | | | | | |
|---|---|---|---|---|---|---|---|---|---|---|
| | (mg · kg⁻¹ · | Week | Week | Week | Week | Week | Week | Week | Week | Week |
| Group | d⁻¹) | 0 | 1 | 2 | 3 | 4 | 5 | 6 | 7 | 8 |
| Normal | — | 196.29 ± | 236.66 ± | 368.89 ± | 397.12 ± | 411.60 ± | 429.28 ± | 448.22 ± | 478.01 ± | 497.12 ± |
| control | | 11.43 | 17.36 | 30.91 | 31.58 | 33.49 | 36.81 | 36.46 | 36.37 | 39.37 |
| group | | | | | | | | | | |
| Model | — | 189.21 ± | 213.15 ± | 297.55 ± | 299.35 ± | 308.43 ± | 313.23 ± | 344.95 ± | 351.78 ± | 397.12 ± |
| group | | 12.41 | 12.36 | 36.46* | 22.04* | 25.43* | 22.98* | 44.01* | 26.22* | 30.57* |
| Positive | 50 | 188.19 ± | 211.95 ± | 279.31 ± | 292.58 ± | 317.80 ± | 313.19 ± | 333.57 ± | 360.01 ± | 380.37 ± |
| drug | | 14.62 | 20.75 | 41.05* | 41.80* | 44.63* | 45.51* | 48.82* | 52.79* | 52.88* |
| group | | | | | | | | | | |
| Low dose | 1 | 195.64 ± | 215.16 ± | 282.30 ± | 299.34 ± | 312.99 ± | 317.48 ± | 334.47 ± | 355.89 ± | 375.85 ± |
| group | | 13.22 | 17.62 | 25.51* | 26.51* | 28.23* | 29.75* | 32.73* | 38.97* | 46.00* |
| Medium | 2 | 190.85 ± | 219.44 ± | 274.69 ± | 293.12 ± | 321.78 ± | 328.10 ± | 345.05 ± | 352.90 ± | 383.69 ± |
| dose | | 12.05 | 10.44 | 28.72* | 39.28* | 26.22* | 48.25* | 24.98* | 37.55* | 39.66* |
| group | | | | | | | | | | |
| High dose | 4 | 196.33 ± | 218.24 ± | 285.54 ± | 296.05 ± | 307.43 ± | 321.49 ± | 349.47 ± | 372.49 ± | 392.02 ± |
| group | | 10.82 | 13.72 | 31.80* | 30.67* | 31.52* | 32.43* | 31.48* | 37.88 | 45.54* |

Note: 1. Number of animals: n =16 in each group; 2. Compared with normal control group, in each dose group, *: P < 0.05.

The weight of rats in each group increased gradually, but the weight of rats in each model and administration group was significantly different from that in normal control group since the second week. The difference in weight between alcohol drinking model group and administration groups is not obvious, but the weight gain of high-dose group is higher than that of medium- and low-dose groups, that is, there is a certain relationship between weight gain and administration dosage of 5-methyltetrahydrofolate proposed in the present invention, indicating that 5-methyltetrahydrofolate relieves the loss of appetite caused by alcohol drinking or slow weight gain of rats caused by other factors.

TABLE 5
Improvement of 5-methyltetrahydrofolate calcium salt on alcoholic liver damage and
fatty liver and changes in biochemical indexes of rats tested (x̅ ± s)

| | Dosage | TG | TC | SOD | MDA |
|---|---|---|---|---|---|
| Group | (mg · kg⁻¹ · d⁻¹) | (mmol/L) | (mmol/L) | (U/mgprot) | (nmol/mgprot) |
| Normal control | — | 1.06 ± 0.51 | 1.29 ± 0.31 | 227.21 ± 22.34 | 48.75 ± 9.64 |
| group | | | | | |
| Model group | — | 1.71 ± 0.35* | 1.62 ± 0.67* | 156.85 ± 22.52* | 72.36 ± 11.25* |
| Positive drug | 50 | 1.03 ± 0.34^{#} | 1.45 ± 0.43 | 167.47 ± 18.91 | 70.41 ± 20.33* |
| group | | | | | |
| Low dose | 1 | 1.08 ± 0.34^{#} | 1.42 ± 0.36 | 253.20 ± 24.66^{#} | 45.20 ± 8.17^{#} |
| group | | | | | |
| Medium dose | 2 | 1.03 ± 0.50^{#} | 1.50 ± 0.37 | 257.41 ± 31.28^{#} | 42.17 ± 8.65^{#} |
| group | | | | | |
| High dose | 4 | 1.04 ± 0.43^{#} | 1.47 ± 0.44 | 248.87 ± 34.64^{#} | 47.96 ± 10.28^{#} |
| group | | | | | |

Note:
1. Number of animals: n = 16 in each group;
2. *Compared with the vehicle control group, in each dose group, P < 0.05; ^{#}Compared with the model control group, in each dose group, P < 0.05.

The results show that: the indexes of rats in the model group are significantly different from those in the control group (P<0.05), and TG, MDA and SOD in three dosage group of test substance are significantly different from those in the model group (P<0.05). The 5-methyltetrahydrofolate calcium group at a low dose of 1 mg/kg/day has TG and TC indexes close to those of the positive drug group, and better SOD and MDA indexes than those of the positive drug group, indicating that 5-methyltetrahydrofolate has an excellent therapeutic or relieving effect on liver damage and fatty liver caused by drinking.

TABLE 6
Comparison of inflammatory factors in hippocampus tissue
of rats in each group (x̅ ± s)

| | Dosage | TNF-α | IL-1β |
|---|---|---|---|
| Group | (mg · kg⁻¹ · d⁻¹) | (μg · g⁻¹ prot) | (μg · g⁻¹ prot) |
| Normal control | — | 244.32 ± 29.81 | 60.32 ± 5.32 |
| group | | | |
| Model group | — | 290.45 ± 51.90* | 74.98 ± 7.43 |
| Positive drug | 50 | 286.32 ± 43.79.* | 73.93 ± 9.37 |
| group | | | |
| Low dose | 1 | 250.39 ± 32.40^{#} | 63.05 ± 5.31 |
| group | | | |
| Medium dose | 2 | 241.32 ± 29.32^{#} | 63.24 ± 13.44 |
| group | | | |
| High dose | 4 | 220.43 ± 24.34^{#} | 55.21 ± 3.59^{#} |
| group | | | |

Note:
1. Number of animals: n = 16 in each group;
2. *Compared with normal control group, in each dose group, P < 0.05; ^{#}Compared with model control group, in each dose group, P < 0.05.

The results show that 5-methyltetrahydrofolate calcium can inhibit the production of inflammatory factors induced by chronic alcohol intake in the brain of rats. Individuals are greatly different from each other with large fluctuations in the data of the model group. Therefore, it is necessary to increase duration of alcohol intake and establish a “alcoholism” model to observe the effect of alcohol on the induction of brain inflammation in rats. However, it can be determined that 5-methyltetrahydrofolate can significantly inhibit the expression of inflammatory factors in the brain of rats, showing a certain dose-effect relationship. Therefore, 5-methyltetrahydrofolate can prevent headaches and migraines induced by alcohol.

Pathological Examination:

Control group: clear structure of hepatic lobules, no degeneration, necrosis and hyperplasia of hepatocytes, and no hyperplasia of interstitial connective tissue;

Model group: moderate hyperplasia of hepatic interstitial connective tissue and fatty degeneration of hepatocytes; low-dose group: a few fatty vacuoles in hepatocytes, and no obvious hyperplasia of interstitial connective tissue; positive drug group, medium-dose group and high-dose group: no hyperplasia of interstitial connective tissue and fatty degeneration of hepatocytes. (Refer to accompanying drawings 1-6 to the disclosure)

Experiment 5 Effect of 5-Methyltetrahydrofolate Calcium on Hyperlipemia Rats

50 SPF-grade SD rats were chosen in half female and half male and fed with normal diet for 5 weeks, then randomly divided into normal diet feeding group (n=9) and model group (n=41), which were respectively fed with normal diet or high-fat diet consisting of 83% normal diet, 15% lard, and 2% cholesterol. 7 weeks later, one rat in the control group and one rat in the model group were sacrificed for liver tissue section to check fatty degeneration of hepatocytes (see Table 7) to confirm whether modeling was successful. The remaining 40 modeling rats were randomly divided into low-dose (1 mg·Kg−1) group, medium-dose (2 mg·Kg−1) group, high-dose (4 mg·Kg−1) group, positive drug group, and model group.

Bicyclol (place of origin: Beijing Xiehe Pharmaceutical Factory, specification: 25 mg/tablet, batch number: H20040467) was used as a positive drug.

After successful modeling, rats were administered drugs with dosages as prescribed for 60 consecutive days, continued to be fed with high-fat diet, and dissected on the 61st day. Liver was harvested and weighed, liver index was calculated, right lobe tissue was taken to prepare frozen sections, and fat staining was performed with Sudan III for pathological scoring according to guidelines for diagnosis and treatment of NAFLD. Another liver tissue was taken to prepare homogenate, and total cholesterol (TC), triglyceride (TG), malondialdehyde (MDA), and superoxide dismutase (SOD) in serum were detected. The results are shown in Table 8.

TABLE 7
Comparison of body mass, liver mass and liver index of rats in five groups (x̅ ± s)

| | | | | | Pathological | |
|---|---|---|---|---|---|---|
| | Number | Body mass | Liver mass | | state | |
| | of cases | (g) | (g) | Liver index | (NASH) | |
| Model group | 8 | 475.7 ± 36.5 | 20.6 ± 3.5 | 4.3 ± 0.5 | 4 | cases |
| Positive drug | 8 | 453.8 ± 34.7 | 14.6 ± 4.5 | 3.2 ± 0.4* | 2 | cases |
| group | | | | | | |
| Low dose | 8 | 445.89 ± 39.0 | 15.3 ± 3.5* | 3.4 ± 0.3* | 1 | case |
| group | | | | | | |
| Medium dose | 8 | 452.93 ± 37.5 | 11.6 ± 2.9** | 2.6 ± 0.2** | 0 | case |
| group | | | | | | |
| High dose | 8 | 472.49 ± 37.8 | 10.9 ± 2.5** | 2.3 ± 0.5** | 0 | case |
| group | | | | | | |
| Normal group | 8 | 452.83 ± 37.5 | 13.6 ± 2.8* | 3.0 ± 0.4* | 0 | case |

Note:
*Compared with model group, in each dose group, P < 0.05; **Compared with model group, in each dose group, P < 0.01.

The above data indicate that 5-methyltetrahydrofolate calcium can prevent and treat fatty liver caused by hyperlipidemia, restore the liver to its normal form, and prevent fatty liver in case-control state from changing to nonalcoholic steatohepatitis (NASH). All rats in the medium- and high-dose 5-methyltetrahydrofolate groups exhibited a benign or normal liver.

TABLE 8
Effect of 5-methyltetrahydrofolate calcium on biochemical indexes of (x̅ ± s)

| | Dosage | TG | TC | SOD | MDA |
|---|---|---|---|---|---|
| Group | (mg · kg⁻¹ · d⁻¹) | (mmol/L) | (mmol/L) | (U/mgprot) | (nmol/mgprot) |
| Normal control | — | 1.08 ± 0.53 | 1.27 ± 0.29 | 230.11 ± 21.24 | 49.75 ± 10.54 |
| group | | | | | |
| Model group | — | 1.70 ± 0.27* | 1.92 ± 0.47* | 158.73 ± 21.48* | 74.26 ± 12.05* |
| Positive drug | 50 | 1.23 ± 0.35^{#} | 1.45 ± 0.63 | 157.41 ± 16.45 | 72.39 ± 19.23* |
| group | | | | | |
| Low dose | 1 | 1.07 ± 0.33^{#} | 1.45 ± 0.38 | 234.20 ± 21.36^{#} | 45.20 ± 8.17^{#} |
| group | | | | | |
| Medium dose | 2 | 1.03 ± 0.55^{#} | 1.57 ± 0.36 | 257.41 ± 30.29^{#} | 42.26 ± 9.35^{#} |
| group | | | | | |
| High dose | 4 | 1.05 ± 0.63^{#} | 1.50 ± 0.34 | 260.97 ± 30.04^{#} | 40.98 ± 11.37^{#} |
| group | | | | | |

Note:
Compared with normal control group, in each dose group, *P < 0.05; ^{#}Compared with model control group, in each dose group, P < 0.05.

Table 8 shows that the results are similar to those in Table 5 of Embodiment 4. The liver is an important organ in the metabolism of folic acid, and there is a complex and direct relationship between liver damage and folic acid metabolism. The inventors have found that 5-methyltetrahydrofolate can prevent benign fatty liver from transforming into pathological fatty liver, and can produce therapeutic effect under a certain dosage.

Experiment 6 Concentration of Folic Acid in Plasma of Humans after Drinking Alcohol

Three adults, based on a complete understanding of the experimental protocol, volunteered to participate in the study, with basic information as shown in Table 9.

JK001 capsule: synthetic folic acid, source: Zhengzhou Yuhe Food-Additive Co., Ltd., purity: 99.8%, molecular weight: 441.4, conversion factor: 1. 42 g of synthetic folic acid was taken and mixed evenly with microcrystalline cellulose, filled into 1000 capsules to make JK001 capsules (containing 42 mg folic acid); JK002 capsule: 5-methyltetrahydrofolate calcium, source: Lianyungang Jinkang Hexin Pharmaceutical Co., Ltd., purity: 99.9%, molecular weight: 497.5, conversion factor: 0.8872. 47.34 g of 5-methyltetrahydrofolate calcium was taken and mixed evenly with microcrystalline cellulose, filled into 1000 capsules to make JK002 capsules (containing 42 mg folic acid, conversion value);

JK003 capsule: microcrystalline cellulose, filled into 1000 capsules to make JK003 capsules.

The above capsules have the same model and specifications and cannot be distinguished from each other by the appearance.

Without having any breakfast, the three volunteers took capsules immediately after drinking 400 ml of alcohol in stages (within half an hour), and then ate and drank normally during the 24 h.

TABLE 9
Basic information of volunteers

| | | Weight | | | |
|---|---|---|---|---|---|
| Group | Gender | (kg) | Age | Health condition | Capsule |
| Mr1 | Male | 64 | 23 | Normal liver function, | JK001 |
| | | | | no other diseases | |
| Mr2 | Male | 60 | 25 | Normal liver function, | JK002 |
| | | | | no other diseases | |
| Mr3 | Male | 72 | 25 | Normal liver function, | JK003 |
| | | | | no other diseases | |

Blood was collected from the vein at 30 min before drinking, 0 min before administration, and 0.25, 0.5, 1, 2, 4, 6, 8, 24 h after administration.

6, 8, 24 h. The collected blood samples were centrifuged at 5000 rpm for 5 min at 4° C., and plasma obtained was transferred to a 1.5 mL centrifuge tube, and then stored in a refrigerator at −20° C. The concentration of folic acid in the blood samples was detected according to the analysis and detection conditions in Table 9.

TABLE 10
Analysis and detection conditions

| Liquid phase | Liquid phase: Agilent 1200 HPLC-CTC autosampler | | | | |
|---|---|---|---|---|---|
| conditions | Mobile phase: | | | | |
| | Solution A: methanol-water-formic acid (10:90:0.1, v/v/v) | | | | |
| | Solution B: methanol-formic acid (100:0.1, v/v) | | | | |
| | Time (min) | | | A(%) | B(%) |
| | 0.00 | | | 100.0 | 0.0 |
| | 0.10 | | | 100.0 | 0.0 |
| | 1.00 | | | 10.0 | 90.0 |
| | 2.00 | | | 10.0 | 90.0 |
| | 2.10 | | | 100.0 | 0.0 |
| | 4.00 | | | 100.0 | 0.0 |
| Mass | Injection volume: 5 μL | | | | |
| spectrometry | Flow rate: 0.40 mL/min | | | | |
| conditions | Chromatographic column: Grace Alltima HP C18 (2.1*50 mm) is | | | | |
| | equipped with in-line filter | | | | |
| | AB API4000 tandem mass spectrometer | | | | |
| | Polarity: Cationic model | | | | |
| | Compound-related parameters | | | | |
| | Parameter | DP | EP | CE | CXP |
| | 6S-5- | 80 | 8 | 25 | 9 |
| | methyltetrahydrofolate | | | | |
| | Carbamazepine (IS) | 80 | 8 | 28 | 7 |

| Source-related parameters | |
|---|---|
| GS1 (gas 1) | 50 |
| GS2 (gas 2) | 50 |
| TEM (temperature) | 450 |
| CUR (curtain gas) | 25 |
| IS (spray voltage) | 5500 |
| CAD (collision gas) | 4 |
| ihe (interface heater): | On |

| | |
|---|---|
| Internal standard | Carbamazepine |
| Analyte MRM | Object to be tested: [M + H]+ m/z 460.2→313.2 (quantitative) |
| | [M + H]+ m/z 460.2→331.1 (qualitative) |
| | Internal standard: [M + H]⁺ m/z 237.1→ 193.9 |
| Linear range | Plasma 5~10000 ng/mL |
| Sample | Take 50 μL of blank plasma, add 5 μL of standard working |
| processing | solution of each concentration (add 5 μL of working diluent to |
| | unknown samples and blank samples), vortex for 10 secs, and |
| | then add 5 μL of stabilizer B (DTT and Vc mixed aqueous |
| | solution, 5 mg · mL − 1, this operation is limited to standard curve), |
| | vortex for 10 secs, and then add 300 μL of methanol precipitant |
| | (containing internal standard carbamazepine at 40 ng · mL − 1, no |
| | internal standard carbamazepine is added to double blank |
| | samples) (containing DTT and Vc at the same time, with a |
| | concentration of 1 mg · mL − 1), vortex for 1 min, centrifuge at |
| | 12000 rpm for 10 min, take 20 μL of the supernatant and mix |
| | with 80 μL of stabilizer C (DTT and Vc mixed aqueous solution, |
| | 1 mg · mL − 1), then take 5 μL of the solution for LC-MS/MS |
| | quantitative analysis. |

The results are shown in the table.

TABLE 11
Plasma drug concentration at different blood sampling time

| Blood | Blood concentration | | |
|---|---|---|---|
| sampling | (ng · mL⁻¹) | | |
| time (h) | Mr1 | Mr2 | Mr3 |
| −0.5 | 13.2152 | 12.6531 | 10.1152 |
| 0 | 12.1243 | 11.3575 | 9.5745 |
| 0.25 | 13.1254 | 16.1863 | 8.6253 |
| 0.5 | 14.2134 | 39.3437 | 5.6423 |
| 1 | 12.0134 | 45.0376 | BLOQ |
| 2 | 9.4556 | 49.7386 | BLOQ |
| 4 | BLOQ | 43.1819 | BLOQ |
| 6 | BLOQ | 17.1690 | BLOQ |
| 8 | BLOQ | 10.4056 | BLOQ |
| 24 | 5.1434 | 9.1884 | 5.7857 |

Note:
Linear range: 5~10000 ng/mL; LLOQ: 5 ng/mL; BLOQ: below the limit of quantification

The results show that: heavy drinking can sharply decrease serum folic acid in a short time to the level of LLOQ, which is beyond our expectation. The liver itself can store about 5-20 mg of folic acid, but this portion of folic acid does not enter the bloodstream immediately after drinking alcohol. 5-methyltetrahydrofolate can solve the decrease of serum folic acid caused by heavy drinking in a short time while synthetic folic acid cannot solve this problem.

Combined with the above animal experiment data, it shows that 5-methyltetrahydrofolate calcium has a good protective effect on alcoholic liver injury models, indicating that the lack of folic acid in the body inevitably affects its protective function. This experimental data is consistent with the animal experiment data, which can mutually confirm and complement each other.

Experiment 7 Effect of Sober-Up Capsule on Hangover Symptoms

In order to prove the effect of the present invention on adverse reactions caused by hangovers after drinking, the capsule prepared in Embodiment 3 was used as a sample, and 10 volunteers, aged 20 to 40, male, were randomly divided into two groups, of which one group used the sample, and the other group used negative sample. The two groups took the sober-up sample or negative sample before drinking, and then drank alcohol in a prescribed amount. Before 8:00 am on the next day, they were asked to fill out the questionnaire on whether they had excessive thirst, nausea, drowsiness, sweating, anorexia, fuzzy cognition, migraine and other symptoms. The results are shown in the table.

TABLE 12
Human hangover reaction test results

| | | | Drowsi- | Sweat- | Anor- | Fuzzy |
|---|---|---|---|---|---|---|
| | Thirst | Nausea | ness | ing | exia | cognition |
| Group | (case) | (case) | (case) | (case) | (case) | (case) |
| Negative | 4 | 4 | 3 | 1 | 1 | 0 |
| group | | | | | | |
| Test | 3 | 0 | 0 | 0 | 0 | 0 |
| group | | | | | | |

Note: They are mainly subjective evaluations on corresponding symptoms, i.e. statistical results, which show that, the composition disclosed in the present invention can improve adverse physiological reactions after drinking, especially have an obvious effect on nausea and drowsiness, and can significantly maintain the mental state of drinkers to improve their mood and reduce their drowsiness after drinking.

Experiment 8 Tail Suspension Test and Swimming Test in Mice after Drinking

Clean-grade male Kunming mice, weighing 18-22 g, were chosen. The test was carried out at 23° C. with a humidity of 50%. The mice ate and drank freely, adapting to the environment for 5 days before the test. Then they were divided into 5 groups: model group, normal group, high-dose 5-MTHF-Ca group, medium-dose 5-MTHF-Ca group and low-dose 5-MTHF-Ca group (16 mg·Kg−1, 8mg·Kg−1, 4mg·Kg−1),

10 mice in each group. The model group was given (by gavage) Erguotou (batch number: 201603092, specification: 2L, place of origin: Beijing Shunxin Agriculture Co., Ltd.) at 18 ml·Kg−1, and calcium folate (Lianyungang Jinkang Hexin Pharmaceutical Co., Ltd.). The high-dose, medium-dose and low-dose groups were also given the solution containing Erguotou and calcium folate. The mice were tested after 24 h. The tail suspension test is carried out by fixing the tail of a mouse so that it is suspended head down in a state of desperately struggling to escape but unable to escape. After a period of time, record the immobility time of the mouse in a desperate state in this environment to observe the therapeutic effect after administration. The head of the mouse suspended upside down was 10 cm from the bottom of the box, and two mice were suspended at a time, which were separated by a partition to avoid collision. The mice were suspended for 10 min, and the accumulated immobility time of the suspended mice was counted within the latter 5 min. The results are as follows:

TABLE 13
Tail suspension test in mice (x̅ ± s)

| Group | Suspension immobility time (S) |
|---|---|
| Model group | 89.54 ± 5.58 |
| Normal group | 55.23 ± 1.53** |
| High dose | 63.53 ± 2.98* |
| group | |
| Medium dose | 68.32 ± 3.46* |
| group | |
| Low dose | 72.42 ± 4.52 |
| group | |

Note:
n = 10 *Compared with the model group, p < 0.05, **Compared with the model group, p < 0.01

The mice were placed in a plastic swimming box with a length of 50 cm, a width of 40 cm and a height of 40 cm. The time was counted for 10 min after the mice were placed in the water. The accumulated immobility time of the mice with only slight limb movements and no struggling, and remaining afloat was recorded within the latter 5 min.

TABLE 14
Swimming test in mice (x̅ ± s)

| Group | Swimming immobility time (S) |
|---|---|
| Model group | 143.24 ± 6.53 |
| Normal group | 97.35 ± 3.45** |
| High dose | 104.64 ± 5.24** |
| group | |
| Medium dose | 110.42 ± 2.64** |
| group | |
| Low dose | 123.56 ± 3.64* |
| group | |

Note:
n = 10 *Compared with the model group, p < 0.05, **Compared with the model group, p < 0.01

The results show that the application of the present invention can significantly improve the mental state of mice after drinking alcohol, such as improving the performance of depression in mice.

Experiment 9 Inhibition of 5-Methyltetrahydrofolate and Curcumin on LPS-Induced Inflammatory Response in Astrocytes

The cerebral cortex of Kunming mice at 1˜2 d was taken out under aseptic conditions, washed 3 times with cold PBS after removing residual meninges and blood vessels, prepared into single cell suspension by blowing, centrifuged, the supernatant was discarded, and the precipitant was resuspended in AST basic medium (high-sugar DMEM containing 10% FBS). The suspension was inoculated into a culture flask coated with poly-L-lysine (0.1 mg/ml), and cultured in a CO2 incubator. After 24 h, tissue debris and non-adherent cells were removed by replacing the medium. Afterwards, the medium was replaced every 3 days until the cells reached 80% confluence, then the flask was placed on a constant temperature rotary shaker, where the suspension was centrifuged at 180 r/min, 37° C. for 18 h to remove oligodendrocytes and microglia from the upper layer. Then trypsin digestion was used for subculture. The second-generation AST was inoculated into a 6-well plate, and mice in the experiment was divided into: blank group with PBS; control group:

LPS (1 μg·ml-1), LPS (1 μg·ml-1)+curcumin (10 μg·ml-1), LPS (1 μg·ml-1)+5-MTHF-Ca (10 μg·ml-1); experimental group: LPS (1 μg·ml-1)+curcumin (5 μg·ml-1)+5-MTHF-Ca (5 μg·ml-1). After 24 h, the culture supernatant was collected, and the cytokines IL-6, IL-1β and TNF-α were detected by ELISA. The results are shown in Table 15.

TABLE 15
Effects of curcumin and 5-methyltetrahydrofolate on LPS-stimulated release
of inflammatory factors from microglia (x̅ ± s)

| | IL-6 | IL-β | TNF-α |
|---|---|---|---|
| Group | (pg · ml⁻¹) | (pg · ml⁻¹) | (pg · ml⁻¹) |
| PBS group | 854.82 ± 21.43^{#} | 673.52 ± 13.22^{#} | 483.97 ± 9.38^{#} |
| LPS group | 1178.43 ± 73.02* | 831.93 ± 15.21* | 570.25 ± 10.56* |
| LPS + Cur group | 1003.97 ± 60.33* | 742.12 ± 10.33* | 492.32 ± 8.92^{#} |
| 5-MTHF-Ca group | 993.24 ± 52.05* | 721.45 ± 8.79^{#} | 489.21 ± 10.71^{#} |
| LPS + Cur + | 982.13 ± 45.32*^{#} | 702.53 ± 9.53^{#} | 452.43 ± 8.47^{#} |
| 5-MTHF-Ca group | | | |

Note:
n = 6, compared with normal control group, in each dose group, *P < 0.05; ^{#}Compared with model control group, in each dose group, P < 0.05.

The results show that both 5-methyltetrahydrofolate and curcumin inhibit the lipopolysaccharide-induced release of inflammatory factors from neuroglia, especially for tumor necrosis factor alpha (TNF-α). The combined use of them surprisingly reduces the level of TNF-α below the normal control group, indicating that curcumin and 5-methyltetrahydrofolate have a synergistic effect. This composition inhibits alcohol-induced inflammation in human nervous system, and plays a role in preventing headaches.

Experiment 10 Effects of Drinking and 5-Methyltetrahydrofolate on the Formate in the Urine of SD Rats

In order to study the effects of 5-methyltetrahydrofolate and ethanol on methanol metabolism in rats, SPF-grade female SD rats, fasted overnight, were divided into 4 groups: (1) ethanol group, with intragastric administration of ethanol at a dose of 1 g/kg respectively at 1, 2, 3 h, n=7; (2) control group, with intragastric administration of glucose at a dose of 1.6 g/kg (equivalent to alcohol calories) respectively at 1, 2, and 3 h, n=5; (3) 5-methyltetrahydrofolate group, with intragastric administration of 5-methyltetrahydrofolate calcium at a dose of 4 mg/kg at 0 h, followed by glucose at a dose of 1.6 g/kg (equivalent to alcohol calories) respectively at 1, 2, and 3 h, n=5; (4) 5-methyltetrahydrofolate alcohol group, with intragastric administration of 5-methyltetrahydrofolate calcium at a dose of 4 mg/kg at 0 h, followed by ethanol at a dose of 1 g/kg respectively at 1, 2, and 3 h, n=5.

Food grade alcohol was used, with a methanol content of less than 5 mg/L. Urine was collected, from 0 to 24 h, into a test tube containing 0.1 mL of thioethanol and stored at −70° C. The urine samples were divided into three parts, of which gas chromatographic method, fluorescence method and HPLC-DNPH derivative method were used to detect ethanol and methanol contents (see Table 16), formate content, and formaldehyde content (see Table 17) in urine, respectively.

The method for detecting formate is as follows: 0.1 mL of urine was mixed with 0.1 mL of 10 mmol/L NAD+, 0.1 mL of potassium phosphate buffer (pH 7.4, 20 mmol/L) and 50 μL of formate dehydrogenase, then 0.1 mL of diaphorase (4 U/mL), 50 μL of resazurin solution (0.2 mg/ml) and 0.5 ml of phosphate buffer (pH 6.00, 200 mmol/L) were added. The mixture was incubated at 37° C. for 5 min, then immersed in boiling water for 3 min, and then cooled to room temperature. Fluorescence spectrophotometry was used to determine the content of formate in the mixture, with an emission wavelength of 590 nm and an absorption wavelength of 565 nm.

The method for detecting formaldehyde is as follows: 0.1 ml of urine was taken and filtered through filtering membrane, 0.05 ml of 2,4-dinitrophenylhydrazine (DNPH, 0.1 g/L) and 0.25 ml of trifluoroacetic acid were added to it. The sample was vortexed for 30 s, centrifuged, supernatant 60° C. water bath. HPLC was used for analysis, with a detection wavelength of 355 nm, column temperature of 35° C., and mobile phase of 65% acetonitrile.

TABLE 16
Effects of drinking and 5-methyltetrahydrofolate on
ethanol and methanol contents in urine of rats

| | | 3 h | 4 h | 8 h | 12 h | 16 h | 24 h |
|---|---|---|---|---|---|---|---|
| | Group | (mg · L⁻¹) | (mg · L⁻¹) | (mg · L⁻¹) | (mg · L⁻¹) | (mg · L⁻¹) | (mg · L⁻¹) |
| Ethanol | Ethanol group | 11.2 ± 1.2 | 23.3 ± 1.9 | 25.8 ± 2.5 | 10.3 ± 1.8 | 4.2 ± 1.0 | 1.7 ± 0.8 |
| Content | 5- | 0.6 ± 0.4 | 0.8 ± 0.5 | 1.5 ± 0.4 | 1.5 ± 0.3 | 1.7 ± 0.5 | 1.6 ± 0.4 |
| | methyltetrahydrofolate | | | | | | |
| | group | | | | | | |
| | 5- | 11.6 ± 1.3 | 25.2 ± 2.2 | 24.1 ± 2.3 | 11.8 ± 2.0 | 4.3 ± 0.8 | 1.7 ± 0.7 |
| | methyltetrahydrofolate | | | | | | |
| | ethanol group | | | | | | |
| | Control group | 0.7 ± 0.3 | 0.8 ± 0.4 | 1.4 ± 0.6 | 1.7 ± 0.6 | 1.4 ± 0.5 | 1.5 ± 0.5 |
| Methanol | Ethanol group | 1.7 ± 0.3 | 3.3 ± 0.6 | 7.8 ± 2.1 | 5.6 ± 1.3 | 5.2 ± 1.0 | 4.4 ± 0.7 |
| content | 5- | 1.0 ± 0.3 | 1.1 ± 0.4 | 1.2 ± 0.4 | 1.1 ± 0.4 | 1.3 ± 0.5 | 1.1 ± 0.3 |
| | methyltetrahydrofolate | | | | | | |
| | group | | | | | | |
| | 5- | 1.6 ± 0.3 | 2.5 ± 0.4 | 6.4 ± 1.0 | 4.4 ± 0.9 | 3.7 ± 0.9 | 1.9 ± 0.7 |
| | methyltetrahydrofolate | | | | | | |
| | ethanol group | | | | | | |
| | Control group | 0.9 ± 0.2 | 1.0 ± 0.3 | 1.1 ± 0.2 | 1.1 ± 0.2 | 1.0 ± 0.3 | 1.0 ± 0.2 |

The results show that the content of ethanol in the urine of rats is highest about 8 hours after drinking, and then decreases rapidly. Surprisingly, the content of methanol in the blood of rats after drinking also increases rapidly, consistent with the increasing trend of ethanol. However, the elimination rate of methanol is much slower than that of ethanol. It shows that the metabolism of endogenous methanol is inhibited by ethanol, which leads to an increase in blood methanol concentration. Surprisingly, 5-methyltetrahydrofolate reduced the concentration of methanol in urine.

TABLE 17
Effects of drinking and 5-methyltetrahydrofolate on
formaldehyde and formate contents in urine of rats

| | | 3 h | 4 h | 8 h | 12 h | 16 h | 24 h |
|---|---|---|---|---|---|---|---|
| | Group | (mg · L⁻¹) | (mg · L⁻¹) | (mg · L⁻¹) | (mg · L⁻¹) | (mg · L⁻¹) | (mg · L⁻¹) |
| Formaldehyde | Ethanol group | 0.8 ± 0.6 | 1.8 ± 0.7 | 4.3 ± 1.8 | 6.1 ± 1.8 | 4.2 ± 1.7 | 1.8 ± 0.7 |
| Content | 5- | 0.8 ± 0.4 | 0.8 ± 0.5 | 0.7 ± 0.4 | 0.8 ± 0.3 | 0.8 ± 0.5 | 0.8 ± 0.4 |
| | methyltetrahydrofolate | | | | | | |
| | group | | | | | | |
| | 5- | 0.8 ± 0.4 | 1.7 ± 0.6 | 2.9 ± 1.2 | 3.1 ± 1.0 | 1.9 ± 0.9 | 1.0 ± 0.5 |
| | methyltetrahydrofolate | | | | | | |
| | ethanol group | | | | | | |
| | Control group | 0.8 ± 0.3 | 0.9 ± 0.4 | 0.9 ± 0.5 | 0.9 ± 0.6 | 0.9 ± 0.4 | 1.0 ± 0.5 |
| Formate | Ethanol group | 230 ± 38 | 276 ± 41 | 550 ± 68 | 648 ± 72 | 411 ± 52 | 248 ± 34 |
| content | 5- | 215 ± 33 | 237 ± 42 | 198 ± 36 | 187 ± 32 | 190 ± 41 | 188 ± 36 |
| | methyltetrahydrofolate | | | | | | |
| | group | | | | | | |
| | 5- | 241 ± 42 | 255 ± 30 | 345 ± 43 | 351 ± 45 | 247 ± 48 | 211 ± 39 |
| | methyltetrahydrofolate | | | | | | |
| | ethanol group | | | | | | |
| | Control group | 210 ± 36 | 247 ± 32 | 243 ± 36 | 217 ± 32 | 236 ± 47 | 226 ± 45 |

The results show that the intake of 5-methyltetrahydrofolate can significantly reduce the concentrations of formaldehyde and formate in urine of rats, and promote the elimination of methanol metabolites.

Experiment 11 Effects of Drinking and 5-Methyltetrahydrofolate on the Concentrations of Formaldehyde and Formate in the Brain of Rats

According to the results of Experiment 10, the majority of alcohol in rats is metabolized 24 hours after drinking. The concentrations of formaldehyde and formate, as well as those of serotonin and dopamine in brain tissue of rats were detected at this time. SPF-grade female SD rats, fasted overnight, were divided into 4 groups.

(1) Ethanol group, with intragastric administration of ethanol at a dose of 1 g/kg respectively at 1, 2, 3 h, n=5; (2) control group, with intragastric administration of glucose at a dose of 1.6 g/kg (equivalent to alcohol calories) respectively at 1, 2, and 3 h, n=5; (3) 5-methyltetrahydrofolate group, with intragastric administration of 5-methyltetrahydrofolate calcium at a dose of 4 mg/kg at 0 h, followed by glucose at a dose of 1.6 g/kg (equivalent to alcohol calories) respectively at 1, 2, and 3 h, n=5; (4) 5-methyltetrahydrofolate alcohol group, with intragastric administration of 5-methyltetrahydrofolate calcium at a dose of 4 mg/kg at 0 h, followed by ethanol at a dose of 1 g/kg respectively at 1, 2, and 3 h, n=5.

Food grade alcohol was used, with a methanol content of less than 5 mg/L. At 24 h after drinking (the results of Experiment 10 show that the concentration of ethanol in urine of rats is close to zero after 24 h), eyeballs were removed and blood was collected, rats were sacrificed by spinal dislocation, brain tissue was taken and stored in a freezer at −80° C. Gas chromatography was used to detect methanol and ethanol concentrations in blood of rats, and HPLC was used to detect formaldehyde concentration in brain tissue. 0.1 g of brain tissue was taken and 0.5 ml of SDN lysate was added to make homogenate, then 0.5 ml of trifluoroacetic acid was added. The sample was centrifuged at 4° C., 0.4 ml of supernatant was collected, 0.1 ml DNPH (1 g/L) was added, mixed and incubated in 60° C. water bath for 30 min, centrifuged at 4° C., and supernatant was collected to detect formaldehyde concentration. Fluorescence spectrometry was used to detect formate concentration in brain. Serotonin and dopamine enzyme-linked immunosorbent reagent was used to detect neurotransmitters in brain in strict accordance with the instructions.

The results are as follows:

TABLE 18
Contents of formaldehyde, formate, serotonin, and dopamine
in brain tissue of rats 24 h after drinking

| | | | 5- | |
|---|---|---|---|---|
| | Formaldehyde | Formate | hydroxytryptamine | Dopamine |
| Group | (mg · L⁻¹) | (mg · L⁻¹) | (μg · L⁻¹) | (ng · L⁻¹) |
| Ethanol group | 3.87 ± 0.61* | 32.2 ± 4.7* | 0.07 ± 0.02* | 32 ± 3 |
| 5-methyltetrahydrofolate | 0.39 ± 0.11^{#} | 2.1 ± 0.2^{#} | 0.12 ± 0.04^{#} | 35 ± 3 |
| group | | | | |
| 5-methyltetrahydrofolate | 1.14 ± 0.31*^{#} | 5.5 ± 0.4*^{#} | 0.10 ± 0.1^{#} | 27 ± 4 |
| ethanol group | | | | |
| Control group | 0.48 ± 0.12^{#} | 2.6 ± 0.4^{#} | 0.11 ± 0.03^{#} | 29 ± 4 |

Note:
n = 5, compared with normal control group, in each dose group, *P < 0.05; ^{#}Compared with ethanol control group, in each dose group, P < 0.05.

The results show that 5-methyltetrahydrofolate can promote the metabolism of formaldehyde and formate in brain tissue, and reduce the concentrations of formaldehyde and formate, thereby preventing and treating hangovers. In addition, the level of serotonin in brain tissue of rats after drinking decreased, and 5-methyltetrahydrofolate can improve the secretion of serotonin.

Experiment 12 Effect of 5-Methyltetrahydrofolate on Intestine Induced by Chronic Alcohol

96 SPF-grade SD rats, half female and half male, were chosen with an average weight of 176-220 g at the beginning of the experiment, and randomly divided into vehicle control group (glucose, 10 g·Kg−1) and model control group (liquor, 6 g·Kg−1), 5-methyltetrahydrofolate calcium group (folic acid, 4 mg·Kg−1, glucose, 10 g·Kg−1), half female and half, 16 rats in each group. During the experiment, each rat was given alcohol or isocaloric glucose daily by gavage, and the dose was gradually increased every day. The day on which the dose of alcohol reached 6g·Kg−1 was defined as the first day of modeling. The intestinal permeability of rats was measured at Weeks 2 and 8, then 5 rats were sacrificed at random, blood and liver tissues were taken.

8 weeks later, rats in the remaining groups received fecal microbiota transplantation from patients with alcoholic steatohepatitis, and were sacrificed at Week 10, then blood and liver tissues were taken.

An oral sugar test was carried out to evaluate intestinal permeability. After fasting for 8 h, the rats were given 2.0 ml of sugar solution,

with lactulose (107 mg/kg), mannitol (30 mg/kg), sucralose (15 mg/kg) and sucrose (570 mg/kg). The rats were individually housed in metabolic cages, and urine was collected. The sugar concentration in the urine was determined by gas chromatography. Blood samples were used to analyze endotoxin in serum with Kinetic-QLC kit.

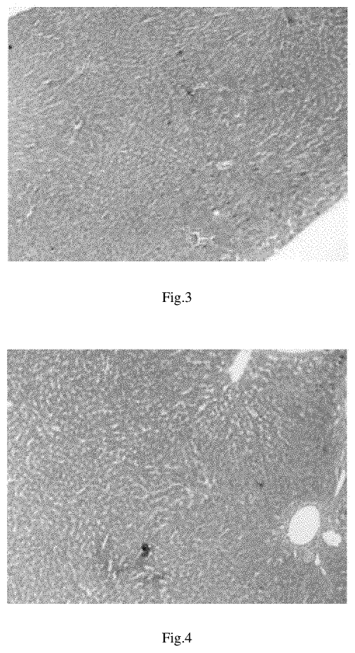

The intestinal permeability detection results are shown in FIGS. 3 and 4. The results show that alcohol at a dose of 6 g/kg destroys the intestinal barrier function of rats, and urine lactulose (small intestinal permeability index) of chronic alcohol-fed rats is significantly higher than that of glucose-fed rats at Week 8. Sucralose (whole intestinal permeability index) in urine of alcohol-fed rats also increased, and the difference was significant at Week 8.

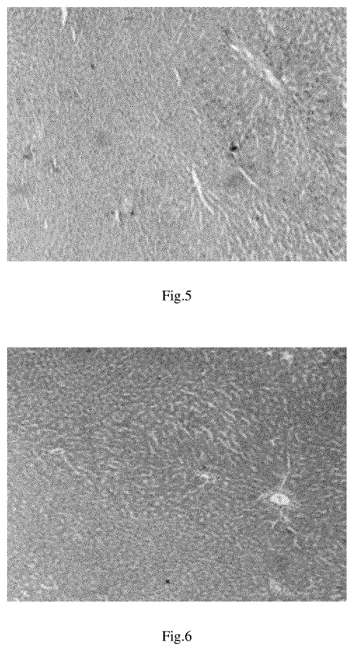

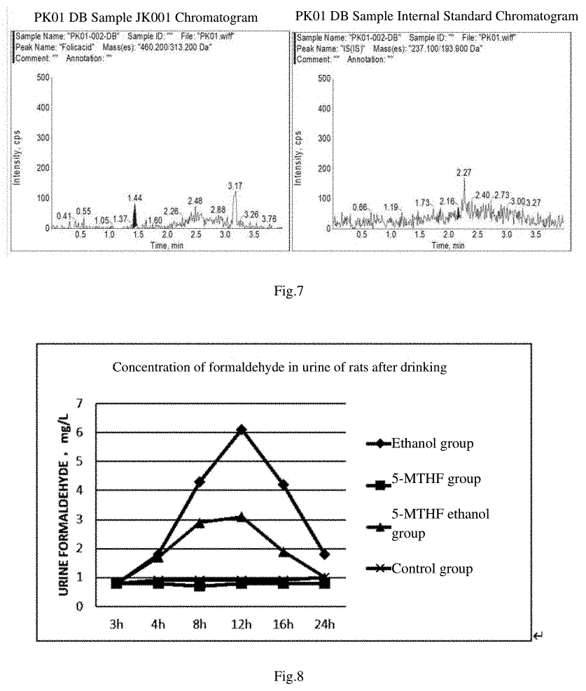

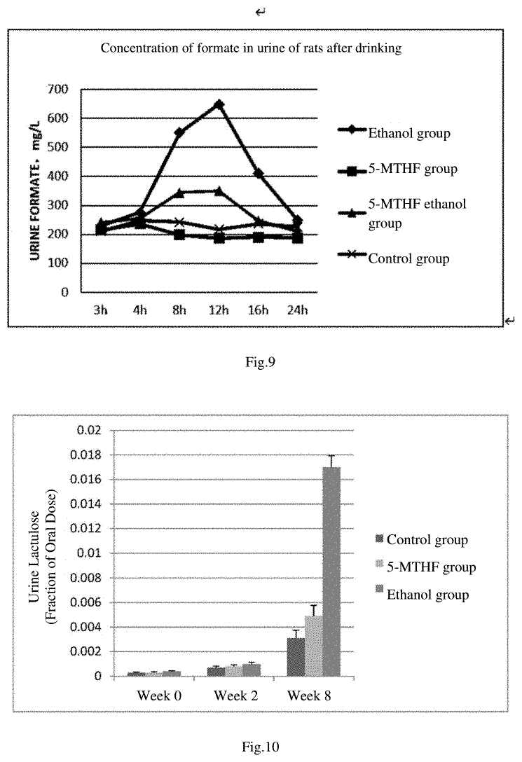

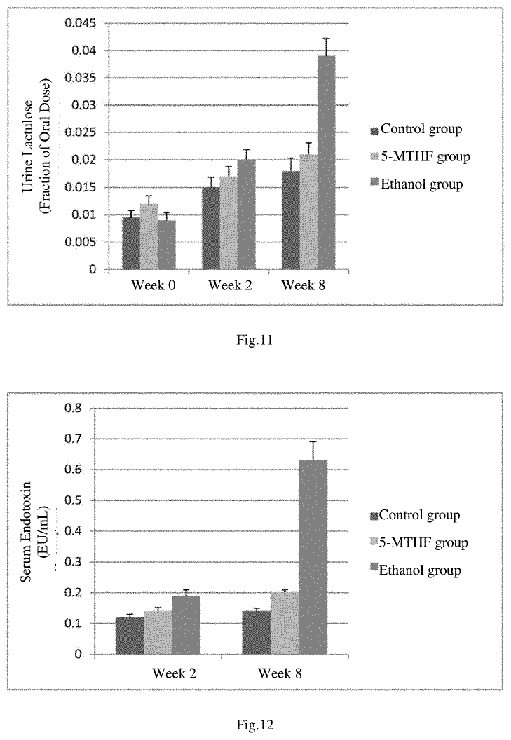

The blood endotoxin detection results are shown in FIG. 5. Endotoxin was detected in blood serum obtained from sacrificed rats. Throughout the study, the value of endotoxin in serum of glucose-fed rats was very low, and drinking led to an increase in serum endotoxin level. The level of endotoxin in the serum of the ethanol group at Week 8 increased by about 3 times compared with that in the second figure.

Fatty degeneration was detected in the liver of sacrificed rats at the earliest within 2 weeks, and alcoholic steatohepatitis (inflammatory cell infiltration, spot necrosis and hepatocyte necrosis) was not seen. Typical symptoms of steatohepatitis were seen in sacrificed rats at Week 8, but still few. It has been determined that endotoxin is an important factor that causes severe liver injury and promotes the development of hepatitis. Endotoxin produced by bacteria in intestinal lumen penetrates into the hepatic portal circulation and then reaches the liver.

60 g of stools were collected from patients with alcoholic steatohepatitis, mixed with 300 mL of normal saline and filtered, and the bacteria solution was stored in an anaerobic bag at 4° C. for later use. After 8 weeks, the remaining rats in all groups were anesthetized with ether, and catheter was used to slowly inject the bacteria solution (2 mL) into their colon, and the rats continued to be fed with the same doses the next day. At Week 10, all rats were sacrificed, liver tissues were stained with H&E and sectioned for disease assessment. At least three different sections of each rat were studied to reasonably assess liver lesions. In order to reasonably assess fatty degeneration, necrosis, inflammation, and fibrosis of liver, liver lesions were graded into different degrees. The proportion of fatty liver cells was <50%, 50-75% and >75%, respectively, corresponding to the severity of fatty degeneration. Focal necrosis was also quantified (number of necrotic foci/mm2), and dense inflammatory infiltrates were also graded. Alcoholic steatohepatitis (ASH) is defined as the presence of inflammatory cell infiltration, spot necrosis and stem cell necrosis in the liver.

The results are as follows:

TABLE 19
Indexes of hepatitis and stem cell damage after transplantation of intestinal
bacteria in different groups

| | | Ethanol group | | 5-methyltetrahydrofolate | | Control group | |
|---|---|---|---|---|---|---|---|
| | | Case percentage | | group | | Case percentage | |
| | | Week | Week | Case percentage | | Week | Week |
| | | 8 | 10 | Week 8 | Week 10 | 8 | 10 |
| Check item | | (n = 5) | (n = 6) | (n = 5) | (n = 6) | (n = 5) | (n = 6) |
| Fatty | Percentage of fatty | 20% | 100% | 0% | 0% | 0% | 0% |
| degeneration | liver cells > 75% | | | | | | |
| | Percentage of fatty | 40% | 0% | 20% | 33.3% | 20% | 20% |
| | liver cells 50-75% | | | | | | |
| | Percentage of fatty | 40% | 0% | 80% | 66.7% | 80% | 80% |
| | liver cells < 50% | | | | | | |
| Necrosis foci | >3 pcs/mm² | 0% | 66.7% | 0% | 0% | 0% | 0% |
| | 1-3 pcs/mm² | 20% | 33.3% | 0% | 0% | 0% | 0% |
| | <1 pc/mm² | 80% | 0% | 100% | 100% | 100% | 100% |
| Liver fibrosis | | 0% | 1 case | 0% | 0% | 0% | 0% |
| | | | of mild | | | | |
| | | | peri- | | | | |
| | | | sinusoid | | | | |
| | | | fibrosis | | | | |
| Inflammatory | >4 pcs/mm² | 0% | 50% | 0% | 0% | 0% | 0% |
| foci | 1-4 pcs/mm² | 40% | 50% | 0% | 0% | 0% | 0% |
| | <1 pc/mm² | 60% | 0% | 100% | 100% | 100% | 100% |

The results support that intestinal endotoxin infiltration is involved in the occurrence of hepatic necrosis and inflammation, which is consistent with previous conclusions. Alcohol and endotoxin exert a synergistic damaging effect on the liver. Oral non-absorbable antibiotics or lactic acid bacteria can reduce liver injury in rats caused by alcohol. Experiments have also proved that endotoxemia precedes steatohepatitis.

The transplantation of intestinal microbiome from patients with severe alcoholic steatohepatitis was used to humanize normal rats to induce liver injury in rats. The results show that 5-methyltetrahydrofolate can improve intestinal barrier function in the presence of alcohol and prevent liver injury caused by harmful bacteria.

What is claimed is:

1. A pharmaceutical composition or health-care food composition comprising an effective amount of 5-methyltetrahydrofolate, and the composition is used to treat or relieve diseases or symptoms caused by drinking or alcohol, wherein the dosage of 5-methyltetrahydrofolate in the composition is 5-50 mg/day.

2. The composition according to claim 1, wherein it is used to treat, relieve or prevent injuries or diseases or symptoms caused by acute alcohol intoxication including headaches caused by drinking, negative emotions or depression caused by drinking, and hangover symptoms or combinations thereof after drinking.

3. The composition according to claim 1, wherein it is used to treat, relieve or prevent injuries or diseases caused by chronic alcohol intoxication including alcoholic fatty liver, central nervous system (CNS) inflammation and migraines and headaches caused by it.

4. The composition according to claim 1, wherein it is used to treat or relieve non-alcoholic fatty liver disease including non-alcoholic fatty liver (NAFL) and non-alcoholic steatosis hepatitis (NASH).

5. The composition according to claim 1, wherein it is used to shorten sobering up time or reduce risk of cardiovascular and cerebrovascular diseases caused by drinking.

6. The composition according to claim 1, wherein it can significantly reduce the levels of total cholesterol (TC), triglycerides (TG), and malondialdehyde (MDA) in serum and increase superoxide dismutase (SOD) level.

7. The composition according to claim 1, wherein it can treat fatty liver, transform pathological steatohepatitis into benign fatty liver, and prevent pathological process of liver fibrosis and cirrhosis.

8. The composition according to claim 2, wherein 5-methyltetrahydrofolate reduces the production of endogenous methanol and/or formaldehyde, and/or promotes the metabolism of formaldehyde and formate in brain tissue, thereby reducing the concentrations of formaldehyde and formate to prevent and treat hangovers.

9. The composition according to claim 2, wherein 5-methyltetrahydrofolate improves the secretion of 5-hydroxytryptamine (5-HT) to prevent and treat negative emotions or depression caused by drinking.

10. A method of using 5-hydroxytryptamine or the composition comprising an effective amount of 5-methyltetrahydrofolate, wherein the dosage of 5-methyltetrahydrofolate in the composition is between 5-50 mg/day to claim 1 in the preparation of medicines for preventing and treating the diseases or symptoms including diseases or symptoms caused by acute alcohol intoxication including headaches caused by drinking, negative emotions or depression caused by drinking, hangover symptoms or combinations thereof after drinking, injuries or diseases caused by chronic alcohol intoxication including alcoholic fatty liver, central nervous system (CNS) inflammation and migraines and headaches caused by it, non-alcoholic fatty liver disease including non-alcoholic fatty liver (NAFL) and non-alcoholic steatosis hepatitis (NASH), reduce risk of cardiovascular and cerebrovascular diseases caused by drinking, to reduce the levels of total cholesterol (TC), triglycerides (TG), and malondialdehyde (MDA) in serum and increase superoxide dismutase (SOD) level, to transform pathological steatohepatitis into benign fatty liver, and prevent pathological process of liver fibrosis and cirrhosis, to reduce the production of endogenous methanol and/or formaldehyde, and/or promote the metabolism of formaldehyde and formate in brain tissue, thereby reducing the concentrations of formaldehyde and formate, or to improve the secretion of 5-hydroxytryptamine (5-HT).

11. The composition according to claim 1, further comprising other active compounds that act alone or synergistically including an effective amount of curcumin, wherein the mass ratio of 5-methyltetrahydrofolate to curcumin is 3˜1:1˜100.

12. The composition of claim 11, wherein the mass ratio of 5-methyltetrahydrofolate to curcumin is 3˜1:1˜3.

13. The composition of claim 12, wherein the mass ratio of 5-methyltetrahydrofolate to curcumin is 1:1.

14. The composition of claim 4, wherein the composition protects intestinal barrier function, reduces serum endotoxin levels, and prevents production of alcoholic “permeable intestine” thus to reduce the flow of endotoxins into enterohepatic circulation.

15. The composition of claim 6, wherein it is h used to treat or prevent hyperlipidemia and diseases caused by hyperlipidemia.
